(19) United States
(12) Patent Application Publication
(10) Pub. No.: US 2026/0250756 A1
(43) Pub. Date: Aug. 27, 2026

(54) DYNAMIC MELT DETECTION

(71) Applicant: BioFire Diagnostics, LLC, Salt Lake City, UT (US)

(72) Inventors: Jessica K. Baer, Cottonwood Heights, UT (US); David Duba, Salt Lake City, UT (US); Andrew Hemmert, Salt Lake City, UT (US); Jay D. Jones, Salt Lake City, UT (US); Thomas Charles Robbins, Salt Lake City, UT (US); James Nicholas Francis, Salt Lake City, UT (US)

(21) Appl. No.: 19/107,504

(22) PCT Filed: Aug. 29, 2023
(86) PCT No.: PCT/US2023/073089

§ 371 Date: Feb. 27, 2025

Related U.S. Application Data
(60) Provisional application No. 63/374,350, filed on Sep. 1, 2022.

(51) Int. Cl. C12Q 1/686; C12Q 1/689; C12Q 1/6895; G01N 21/64

(52) U.S. Cl. CPC C12Q 1/686; C12Q 1/689; C12Q 1/6895; G01N 21/6428; G01N 21/6452; C12Q 2600/156

(57) ABSTRACT

The present disclosure relates to methods and systems suitable for simultaneously amplifying a number of potential targets and then performing a melt detection based on whether or not any amplification is detected for any target nucleic acid(s), wherein the melt parameters are limited by the melt temperature range characteristic for the target nucleic acid(s) for which amplification is detected. This invention retains the resolution achieved by nucleic acid melts with a slow, steady ramp rate between the annealing and denaturation temperatures, but does so faster by using faster ramp rates at temperatures where no melt signature is expected from the reaction, and slower at temperatures where a melt signature is expected from the reaction.

### CROSS-REFERENCE TO RELATED APPLICATIONS

This application claims the benefit of and priority to U.S. Prov. Pat. App. No. 63/374,350 filed 1 Sep. 2022, the entirety of which is incorporated herein by reference.

### BACKGROUND OF THE INVENTION

In the United States, Canada, and Western Europe infectious disease accounts for approximately 7% of human mortality, while in developing regions infectious disease accounts for over 40% of human mortality. Infectious diseases lead to a variety of clinical manifestations. Among common overt manifestations are fever, pneumonia, meningitis, diarrhea, and diarrhea containing blood. While the physical manifestations suggest some pathogens and eliminate others as the etiological agent, a variety of potential causative agents remain, and clear diagnosis often requires a variety of assays to be performed. Traditional microbiology techniques for diagnosing pathogens can take days or weeks, often delaying a proper course of treatment.

In recent years, the polymerase chain reaction (PCR) has become a method of choice for rapid diagnosis of infectious agents. PCR can be a rapid, sensitive, and specific tool to diagnose infectious disease. A challenge to using PCR as a primary means of diagnosis is the variety of possible causative organisms and the low levels of organism present in some pathological specimens. It is often impractical to run large panels of PCR assays, one for each possible causative organism, most of which are expected to be negative. The problem is exacerbated when pathogen nucleic acid is at low concentration and requires a large volume of sample to gather adequate reaction templates. In some cases, there is inadequate sample to assay for all possible etiological agents. A solution is to run “multiplex PCR” wherein the sample is concurrently assayed for multiple targets in a single reaction. While multiplex PCR has proven to be valuable in some systems, shortcomings exist concerning robustness of high-level multiplex reactions and difficulties for clear analysis of multiple products. To solve these problems, the assay may be subsequently divided into multiple secondary PCRs. Nesting secondary reactions within the primary product often increases robustness. However, this further handling can be expensive and may lead to contamination or other problems.

The FilmArray® (BioFire Diagnostics, Inc., Salt Lake City, UT) is a user friendly, highly multiplexed PCR system developed for the diagnostic market. The single sample instrument accepts a diagnostic “pouch” that integrates sample preparation and nested multiplex PCR. Integrated sample preparation provides ease-of-use, while the highly multiplexed PCR provides both the sensitivity of PCR and the ability to test for many organisms simultaneously (e.g., up to 30 or more different organism and/or molecular markers). This system is well suited to pathogen identification where a number of different pathogens all manifest similar clinical symptoms. Current available diagnostic panels include a respiratory panel for upper respiratory infections, a blood culture panel for blood stream infections, a gastrointestinal panel for GI infections, a meningitis/encephalitis panel for central nervous system infections, a pneumonia panel for lower respiratory infections, and a bone and joint panel for bone and joint infections. Other panels are in development.

PCR may be conceptually divided into 3 reactions, each usually assumed to occur over time at each of three temperatures. Such an “equilibrium paradigm” of PCR is easy to understand in terms of three reactions (denaturation, annealing, and extension) occurring at 3 temperatures over 3 time periods each cycle. However, this equilibrium paradigm does not fit well with physical reality. Instantaneous temperature changes do not occur; it takes time to change the sample temperature, and temperature may not be homogeneous throughout the sample, particularly where larger volumes are used. Furthermore, individual reaction rates vary with temperature, and once primer annealing occurs, polymerase extension immediately follows. More accurate, particularly for rapid PCR, is a kinetic paradigm where reaction rates and temperature are always changing. Holding the temperature constant during PCR is not necessary as long as the products denature and the primers anneal. Under the kinetic paradigm of PCR, product denaturation, primer annealing, and polymerase extension may temporally overlap and their rates continuously vary with temperature. Under the equilibrium paradigm, a cycle is defined by 3 temperatures each held for a time period, whereas the kinetic paradigm requires transition rates and target temperatures.

When PCR was first popularized in the late 1980s, the process was slow. A typical protocol was one minute for denaturation at 94° C., two minutes for annealing at 55° C., and three minutes for extension at 72° C. When the time for transition between temperatures was included, 8-minute cycles were typical, resulting in completion of 30 cycles in four hours. Twenty-five percent of the cycling time was spent in temperature transitions. As cycling speeds increased, the proportion of time spent in temperature transitions also increased and the kinetic paradigm became more and more relevant. During rapid cycle PCR, the temperature is usually changing. For rapid cycle PCR of short products (<100 bps), 100% of the time may be spent in temperature transition and no holding times are necessary. For rapid cycle PCR of longer products, a temperature hold at an optimal extension temperature may be included.

One way to decrease cycle time is to introduce variations to the PCR protocol to ease the temperature cycling requirements. Over the years, systems have become faster, and the kinetic requirements of denaturation, annealing, and extension have become clearer. Shorter cycling times, reducing 3-step cycling (denaturation, annealing, and extension) to 2-steps (denaturation and a combined annealing/extension step), longer primer lengths, shorter product lengths, and the like can reduce PCR reaction time while maintaining high diagnostic accuracy. Even with protocol variations, many diagnostic PCR reactions conclude with a confirmatory melt detection step. DNAs have a melt temperature range that is specific to their length and A-T/G-C composition. Probe-free DNA melting analysis generally depends on the fact that DNA binding dyes fluoresce strongly in the presence of double-stranded DNA and fluoresce weakly or not at all in the presence of single-stranded DNA. Thus, for a given amplification reaction, the expected product will have an expected melt temperature range that is characteristic of that product and, as such, melting analysis can be used to confirm that the expected product was made in the amplification reaction.

However, as PCR gets faster, the time spent in melting becomes an increasingly larger portion of the run time. One possible solution would be to ramp the temperature faster during melting, but it has been found that melting curves generated by faster temperature ramping often result in decreased sensitivity to amplicon differences. It would be desirable to generate melting curves using faster temperature ramping while maintaining sensitivity to amplicon differences. There exists a need in the art for retaining the melt resolution achieved by the slower temperature ramp rates while also shortening the time needed for melt analysis.

### SUMMARY OF THE INVENTION

The present disclosure relates to methods and systems suitable for simultaneously amplifying a number of potential targets and then performing a melt detection based on whether or not any amplification is detected for any target nucleic acid(s), wherein the melt parameters are limited by the melt temperature range characteristic for the target nucleic acid(s) for which amplification is detected. High resolution DNA melting has traditionally been conducted using a fixed temperature ramp rate that encompasses the entire range of temperatures over which the reaction products are expected to melt. This invention retains the resolution achieved by a slower temperature ramp rate but does so in less time by using faster melt ramp rates at temperature ranges where no melt signature is expected from the reaction, and slower melt ramp rates at temperature ranges where a melt signature is expected from the reaction. These variable melt ramp ranges and rates are defined by the predicted or experimentally determined melt temperature(s) of the amplicon(s) for which amplification is actually detected so that time is not spent ramping slowly in a large temperature window for all possible targets.

For example, a test (e.g., a panel including a number of assays for a number of pathogens) may be designed to amplify and detect 20 or more targets. If, in a specific use case, only one or two targets are present in a sample, the melt parameters can be dynamically set based on the predicted or experimentally determined melt temperature(s) of the target amplicon(s) present (referred to herein as the range of interest or ROI) instead of having one large melt range and one slow melt ramp rate designed to capture the melts of all of the possible targets. For example, as will be explained in greater detail herein below, the melt temperature may be ramped rapidly (greater than 4° C./sec, e.g., 6-20° C./sec) in a first temperature range where no melt of the detected the target amplicon(s) is expected, ramped slowly (e.g., less than 4° C./sec, 0.01-2° C./sec, 1-2° C./sec) through the ROI, and then again ramped rapidly (greater than 4° C./sec, e.g., 12-20° C./sec) after the ROI up to the denaturation temperature. This fast-slow-fast ramping protocol can save time while preserving the resolution of a traditional slow melt. If amplification of different target amplicons is detected in more than one well, this ‘fast-slow-fast’ protocol suitably may be used if the amplification of the different target amplicons is detected at the same or nearly the same time and if the melt ranges of the target amplicons sufficiently overlaps. In another aspect, a modified ‘fast-slow-fast-slow-fast’ protocol suitably may be used if the amplification of the different target amplicons is detected at the same or nearly the same time and if there is sufficient separation between the melt regions of interest between the two organisms suspected to be in the sample. In one aspect, the systems described herein suitably may be designed to determine which is the fastest and most efficient melt protocol for situations where amplification of the different target amplicons is detected at the same or nearly the same time (e.g., ‘fast-slow-fast’ vs. ‘fast-slow-fast-slow-fast’).

The invention described herein suitably may include performing a melt detection after a fixed number of PCR cycles if amplification is detected (e.g., the fluorescence signal in a sample well rises above a threshold value) for one or more target nucleic acids. The invention described herein suitably may include performing a melt detection at any point in the reaction if amplification is detected for one or more target nucleic acids. The invention described herein suitably may include not performing a melt detection if no amplification is detected for any target nucleic acid(s).

The invention described herein suitably may include a method for identifying which of a plurality of target nucleic acids is in a sample (e.g., from a plurality of organisms). The method suitably may include steps of providing the sample (e.g., a respiratory sample, a blood sample, a positive blood culture sample, or the like) suspected of containing at least one target organism, and providing a plurality of sample wells, each sample well provided with a portion of the sample and primers for amplifying a target nucleic acid sequence from a different one of the plurality of target nucleic acids. Each target nucleic acid from each different organism and/or molecular marker has a melt temperature range that is characteristic for that target nucleic acid. The method suitably may further include providing a fluorescent dye that produces a rising fluorescence signal in response to a rise in concentration of nucleic acid in the plurality of sample wells, and simultaneously subjecting the plurality of sample wells to amplification conditions for a selected number of cycles. If during the amplification conditions a sample well shows positive nucleic acid amplification, then the method suitably may include performing a melt detection, wherein the melt detection is defined by the melt temperature range and melt ramp rate characteristic for the target nucleic acid for detection of the organism in the sample well.

The invention described herein suitably may include a method for identifying which of a plurality of target nucleic acids and/or molecular markers are in a sample. The method suitably may include steps of providing the sample suspected of containing at least one target organism or molecular marker of the plurality of organisms and/or molecular markers and providing a plurality of sample wells configured for amplification of the plurality of target nucleic acids. Each sample well of the plurality of sample wells suitably may include a pair of primers for amplification of one of the plurality of target nucleic acids. In some embodiments, each sample well of the plurality of sample wells suitably may include more than one pair of primers for amplification of more than one of the plurality of target nucleic acids. Each target nucleic acid suitably may have a characteristic melt temperature range and a melt ramp rate. The method suitably may further include providing a fluorescent dye that produces a rising fluorescence signal in response to a rise in concentration of nucleic acid in the plurality of sample wells, distributing the sample amongst the plurality of sample wells such that each sample well contains a portion of the sample, simultaneously subjecting the plurality of sample wells to amplification conditions, wherein the amplification conditions include repeated thermal cycles each comprising a primer annealing step, a primer elongation portion, and a nucleic acid denaturation step, and acquiring during the thermal cycles the fluorescence signal in each of the plurality of sample wells. If the fluorescence signal of a sample well meets the conditions to indicate that the expected DNA target is presented in the sample (as an example the fluorescence signal in a sample well is greater than a threshold value, the threshold being greater or equal than a limit-of-detection value for concluding a rise in the concentration of nucleic acid in the sample well), then the method suitably may include performing a melt detection, wherein the melt detection is defined by the melt temperature range and melt ramp rate characteristic for the target nucleic acid for detection of the organism in the sample well in which amplification above the threshold value was detected.

The invention described herein suitably may include a method for fluorescent detection of a nucleic acid. The method suitably may include steps of providing a sample suspected of containing at least one of a plurality of organism, providing a plurality of sample wells, each sample well provided with primers for amplifying a target nucleic acid from a different one of the plurality of organisms, moving a portion of the sample into each of the plurality of sample wells, simultaneously subjecting the plurality of sample wells to amplification conditions, and acquiring during the thermal cycles the fluorescence signal in each of the plurality of sample wells. If the amplitude of the fluorescence signal of a sample well configured for amplification of one target nucleic acid is greater than a threshold value, the threshold being greater or equal than a limit-of-detection value for concluding a rise in the concentration of nucleic acid in the sample well, then the method suitably may include performing a melt detection, wherein the melt detection is defined by the melt temperature range and melt ramp rate characteristic for the target nucleic acid for detection of the organism in the sample well in which amplification above the threshold value was detected.

The invention described herein suitably may include a system for detecting which of a plurality of target organisms is in a sample. The system suitably may include a vessel comprising a plurality of sample wells, each sample well configured for holding a portion of the sample, nucleic acid primers specific for amplification of one target nucleic acid from one target organism, a fluorescent dye that produces a rising fluorescence signal in response to a rise in concentration of nucleic acid in the plurality of sample wells, and components for amplification, and an instrument configured to concurrently subject the portion of the sample in each of the plurality of sample wells to amplification conditions and then melting conditions. The instrument suitably may include a detector for detecting a fluorescent signal from the fluorescent dye indicating amplification. The instrument suitably may be programmed with thermocycling parameters for amplification of nucleic acids in the plurality of sample wells and with information regarding the target organisms to be amplified in each one of the plurality of wells including melt range information for each of the target nucleic acids from the target organisms. The instrument may suitably be further programmed to monitor fluorescence in the plurality of wells during the amplification conditions and, if during the amplification conditions, a sample well shows an increase in fluorescence indicative of positive nucleic acid amplification, then performing a melt detection, wherein the melt detection is defined by the melt temperature range and melt ramp rate for the target nucleic acid amplified in the sample well.

What is described is:
A1. A method for identifying which of a plurality of target nucleic acids is in a sample, comprising
providing the sample suspected of containing at least one target nucleic acid,
providing a plurality of sample wells, each sample well provided with a portion of the sample and primers for amplifying a target nucleic acid sequence from a different one of the plurality of target nucleic acids, wherein each target nucleic acid sequence has a characteristic melt temperature range,
providing a fluorescent dye that produces a rising fluorescence signal in response to a rise in concentration of nucleic acid in the plurality of sample wells,
simultaneously subjecting the plurality of sample wells to amplification conditions for a selected number of cycles,
determining if a sample well exhibits positive nucleic acid amplification as evidenced by a rising fluorescence signal from the sample well during the amplification conditions, and
responsive to determining that the sample well exhibits positive nucleic acid amplification, performing a melt detection configured to detect the target nucleic acid amplified in the sample well, wherein the melt detection is defined by the melt temperature range characteristic for the target nucleic acid in the sample well.
A2. The method of clause A1, wherein positive nucleic acid amplification is determined by the fluorescence signal in the sample well rising above a threshold.
A3. The method of clause A1 or A2, wherein the threshold is one of a crossing point (Cp), a Cp plus one or more additional cycles of amplification, Ct, relative florescence units rising above a threshold value, mathematical modeling, signal processing, and combinations thereof.
A4. The method of any one of clauses A1-A3, further comprising analyzing the fluorescent signal of the plurality of wells in real time to determine if amplification has occurred in a sample well and performing the melt detection if amplification is determined to have occurred in the well, wherein a temperature range for the melt detection is limited by a known melt temperature range for the target nucleic acid in the well.
A5. The method of any one of clauses A1-A4, further comprising not performing a melt detection if no sample well shows positive nucleic acid amplification.
A6. The method of any one of clauses A1-A5, wherein the plurality of sample wells includes one or more control wells and wherein a melt detection is not performed if only the one or more control wells show positive nucleic acid amplification.
A7. The method of any one of clauses A1-A6, wherein the melt detection comprises:
a first ramp rate during a first portion of the melt,
a second ramp rate during a second portion of the melt, and
a third ramp rate during a third portion of the melt,
the second ramp rate being slower than the first and third ramp rates and wherein
the second portion of the melt is defined by the melt temperature range characteristic for the target nucleic acid for detection of the organism in the sample well.
A7.1. The method of any one of clauses A1-A7, wherein the third ramp rate is faster than the first ramp rate.
A7.2. The method of any one of clauses A1-A7.1, wherein the second ramp rate is in a range of 0.01 to 4° C./sec, or, preferably, 0.01 to 2° C./sec.
A7.3. The method of any one of clauses A1-A7.2, wherein the second ramp rate is preferably no greater than 0.5° C./sec, more preferably no greater than 1° C./sec, and most preferably less than or equal to 2° C./sec.
A7.4. The method of any one of clauses A1-A7.3, wherein the second ramp rate is adapted to detect a nucleic acid melting signature indicative of a genotype, a sequence variant, or a genetic mutation in a target nucleic acid that alters one or more of a nucleic acid melt temperature or a shape of a nucleic acid melting curve relative to the target nucleic acid without the genotype, sequence variant, or genetic mutation.
A7.5. The method of any one of clauses A1-A7.4, further comprising detecting a single nucleotide polymorphism (SNP).
A7.7. The method of any one of clauses A1-A7.5, further comprising detecting an antimicrobial resistance (AMR) marker.
A7.8. The method of any one of clauses A1-A7.7, further comprising detecting presence of an organism via a first melt detection in first assay and, if the organism is detected as present in the first assay, performing a second melt detection in a second assay to detect presence of or absence of the genotype, sequence variant, or genetic mutation.
A8. The method of any one of clauses A1-A7.8, wherein a positive or negative call in a sample well determines whether a melt detection is performed and determines the temperature range of the second portion of the melt.
A8.1. The method of any one of clauses A1-A8, wherein the temperature range of the second portion of the melt is the melt temperature range for the target amplicon, the melt temperature range for the target amplicon+/−0.5° C. to 10° C., preferably the melt temperature range for the target amplicon+/−2° C. to 6° C.
A9. The method of any one of clauses A1-A8.1, wherein two or more wells show a fluorescent signal indicative of positive amplification.
A10. The method of any one of clauses A1-A9, further comprising performing one melt detection with one melt temperature range for the target nucleic acids amplified in the two or more wells.
A11. The method of any one of clauses A1-A10, further comprising performing a first melt detection with a first melt temperature range characteristic for melting the target nucleic acid amplified in the first well and performing at least a second melt detection with a second melt temperature range characteristic for melting the target nucleic acid amplified in the second well.
A11.1. The method of any one of clauses A1-A11, wherein the melt detection comprises one of:
a first ramp rate during a first portion of the melt,
a second ramp rate during a second portion of the melt, and
a third ramp rate during a third portion of the melt,
the second ramp rate being slower than the first and third ramp rates and wherein the second portion of the melt is defined by the melt temperature ranges characteristic for the target nucleic acids in the first and second sample wells, or
a first ramp rate during a first portion of the melt,
a second ramp rate during a second portion of the melt,
a third ramp rate during a third portion of the melt,
a fourth ramp rate during a fourth portion of the melt, and
a fifth ramp rate during a fifth portion of the melt,
the second and fourth ramp rates being slower than the first, third, and fifth ramp rates and wherein the second portion of the melt is defined by the melt temperature range characteristic for the target nucleic acid in the first sample well and the fourth portion of the melt is defined by the melt temperature range characteristic for the target nucleic acid in the second sample well.
A11.2. The method of any one of clauses A1-A11.1, wherein the second ramp rate is in a range of 0.05 to 4° C./sec, or the second and fourth ramp rates are each in a range of 0.05 to 4° C./sec.
A11.3. The method of any one of clauses A1-A11.2, wherein the second ramp rate or the second and fourth ramp rates are preferably no greater than 0.5° C./sec, more preferably no greater than 1° C./sec, and most preferably no greater than 2° C./sec.
A12. The method of any one of clause A1-A11.3, wherein the selected number of cycles of amplification prior to the melt detection is at least 20.
A13. The method of any one of clause A1-A12, wherein the selected number of cycles of amplification prior to the melt detection is assay-specific and determined by the expected concentration of the plurality of target nucleic acids suspected of being in the sample.
A14. The method of any one of clauses A1-A13, further comprising identifying at least one target organism present in the sample by identifying at least one corresponding sample well in which amplification and a melt detection have occurred.
A15. The method of any one of clauses A1-A14, wherein the target nucleic acid can be used for identifying cell-free DNA, a cell, an organism, a molecular marker for anti-microbial resistance, a host-response marker, and combinations thereof.
B1. A method for identifying which of a plurality of target nucleic acids is in a sample, comprising
providing the sample suspected of containing at least one target nucleic acid,
providing a plurality of sample wells configured for amplification of the plurality of target nucleic acids, wherein each sample well of the plurality of sample wells comprises a pair of primers for amplification of one of the plurality of target nucleic acids, and wherein each target nucleic acid has a characteristic melt temperature range,
providing a fluorescent dye that produces a rising fluorescence signal in response to a rise in concentration of nucleic acid in the plurality of sample wells,
distributing the sample amongst the plurality of sample wells such that each sample well contains a portion of the sample,
simultaneously subjecting the plurality of sample wells to amplification conditions, wherein the amplification conditions include repeated thermal cycles each comprising a primer annealing step, a primer elongation portion, and a nucleic acid denaturation step,
acquiring during the thermal cycles the fluorescence signal in each of the plurality of sample wells,
determining that the amplitude of the fluorescence signal of a sample well configured for amplification of one target nucleic acid is greater than a threshold value, the threshold being greater or equal than a limit-of-detection value for concluding a rise in the concentration of nucleic acid in the sample well, and
responsive to determining that the amplitude of the fluorescence signal of the sample is greater than the threshold value, performing a melt detection configured to detect the target nucleic acid amplified in the sample well, wherein the melt detection is defined by the melt temperature range characteristic for the target nucleic acid amplified in the sample well for detection of the organism in the sample well.
B2. The method of clause B1, wherein the threshold value is one of a crossing point (Cp), a Cp plus one or more additional cycles of amplification, Ct, relative florescence units rising above a threshold value, mathematical modeling, signal processing, and combinations thereof.
B3. The method of clause B1 or B2, further comprising monitoring the fluorescent signal of the plurality of wells in real time to determine if amplification has occurred in at least one well of the plurality of sample wells and performing the melt detection if amplification is determined to have occurred in the at least one well, wherein a temperature range for the melt detection is limited by a known melt temperature range for the target nucleic acid in the at least one well.
B4. The method of any one of clauses B1-B3, wherein the timing of the melt detection is determined by the fluorescence signal of one or more wells configured for amplification of the plurality of target nucleic acids rising above the threshold value.
B5. The method of any one of clauses B1-B4, wherein the method does not include performing a set number of thermal cycles prior to performing a melt detection.
B6. The method of any one of clauses B1-B5, further comprising not performing a melt detection if no sample well shows a fluorescence signal above the threshold value.
B7. The method of any one of clauses B1-B6, wherein the plurality of sample wells includes one or more control wells and wherein a melt detection is not performed if only the one or more control wells show a fluorescence signal above the threshold value.
B8. The method of any one of clauses B1-B7, wherein the melt detection comprises:
a first ramp rate during a first temperature range of the melt,
a second ramp rate during a second temperature range of the melt, and
a third ramp rate during a third temperature range of the melt,
the second ramp rate being slower than the first and third ramp rates and wherein the second temperature range of the melt is defined by the melt temperature range of the target nucleic acid amplified in the sample well having the fluorescence signal greater than the threshold value.
B9. The method of any one of clauses B1-B8, wherein the second temperature range of the melt+/−10° C. of the melt temperature range of the target nucleic acid, +/−8° C. of the melt temperature range of the target nucleic acid, +/−6° C. of the melt temperature range of the target nucleic acid, or +/−4° C. of the melt temperature range of the target nucleic acid.
B10. The method of any one of clauses B1-B9, wherein a melt detection is performed and determines the temperature range of the second portion of the melt.
B11. The method of any one of clauses B1-B10, wherein two or more wells show a fluorescent signal greater than the threshold value.
B12. The method of any one of clauses B1-B11, further comprising performing one melt detection with one melt temperature range for the target nucleic acids amplified in the two or more wells.
B13. The method of any one of clauses B1-B12, further comprising performing a first melt detection with a first melt temperature range characteristic for melting the target nucleic acid amplified in the first well and performing at least a second melt detection with a second melt temperature range characteristic for melting the target nucleic acid amplified in the second well.
B14. The method of any one of clauses B1-B13, wherein the plurality of wells are configured for amplification of target nucleic acids sequences from organisms that are present at high titer, organisms present at a lesser titer relative to the organisms present at high titer, and organisms present at low titer relative to the organisms present at lesser titer, and the method further comprising:
performing a first melt detection if one or more wells configured for amplification of the target nucleic acids sequences from organisms that are present at high titer show amplification above the threshold value within 20 or fewer thermal cycles,
performing a second melt detection if one or more wells configured for amplification of the target nucleic acids sequences from organisms that are present at lesser titer show amplification above the threshold value within 25 or fewer thermal cycles, and
performing a third melt detection if one or more wells configured for amplification of the target nucleic acids sequences from organisms that are present at low titer show amplification above the threshold value within 30 or fewer thermal cycles.
B14.1. The method of any one of clauses B1-B14, wherein the first number of thermal cycles is 20 or fewer thermal cycles, the second number of thermal cycles is 25 or fewer thermal cycles, and the third number of thermal cycles is 30 or fewer thermal cycles.
B15. The method of any one of clauses B1-B14.1, further comprising not performing a melt detection if the one or more wells configured for amplification of the target nucleic acids sequences from organisms that are present at high titer show amplification above the threshold value in greater than 20 thermal cycles.
B16. The method of any one of clauses B1-B15, further comprising not performing a melt detection if the one or more wells configured for amplification of the target nucleic acids sequences from organisms that are present at lesser titer show amplification above the threshold value in greater than 25 thermal cycles.
B16.1 The method of any one of clauses B1-B16, further comprising not performing a melt detection if the one or more wells configured for amplification of the target nucleic acids sequences from organisms that are present at the second titer show amplification above the threshold value in greater than the second number of thermal cycles.
B17. The method of any one of clauses B1-B16.1, further comprising not performing a melt detection if none of the one or more wells configured for amplification of the target nucleic acids sequences from organisms that are present at high titer, lesser titer, or low titer show amplification above the threshold value.
B18. The method of any one of clauses B1-B17, further comprising subjecting the sample to multiplex amplification prior to the distributing step.
B19. The method of any one of clauses B1-B18, wherein all steps are performed within a single closed system.
B20. The method of any one of clauses B1-B19, wherein the target nucleic acid can be used for identifying cell-free DNA, a cell, an organism, a molecular marker for anti-microbial resistance, a host-response marker, and combinations thereof.
C1. A method for determining presence of an organism in a sample, comprising
providing a sample suspected of containing at least one of a plurality of organisms,
providing a plurality of sample wells, each sample well provided with primers for amplifying a target nucleic acid from a different one of the plurality of organisms,
moving a portion of the sample into each of the plurality of sample wells,
simultaneously subjecting the plurality of sample wells to amplification conditions,
acquiring during the thermal cycles the fluorescence signal in each of the plurality of sample wells,
determining that the amplitude of the fluorescence signal of a sample well configured for amplification of one target nucleic acid is greater than a threshold value, the threshold being greater or equal than a limit-of-detection value for concluding a rise in the concentration of nucleic acid in the sample well,
responsive to determining that the amplitude of the fluorescence signal of the sample well is greater than the threshold value, performing a melt detection in a melt temperature window to detect the amplified target nucleic acid in the sample well, wherein the melt temperature window is defined by the melt temperature range characteristic for the target nucleic acid, and
responsive to performing the melt detection, determining the presence of the organism in the sample.
C2. The method of clause C1, further comprising subjecting the plurality of sample wells to amplification conditions for a selected number of cycles, and if within selected number of amplification cycles a sample well shows positive nucleic acid amplification, then performing a melt detection, wherein the melt detection is defined by the melt temperature range characteristic for the target nucleic acid for detection of the organism in the sample well.
C3. The method of clause C2, wherein the selected number of cycles of amplification is at least one but 15 or less, at least one but 20 or less, at least one but 25 or less, at least one but 30 or less, or at least one but 35 or less.
C4. The method of any one of clauses C1-C3, further comprising not performing a melt detection if no sample well shows positive nucleic acid amplification.
C5. The method of any one of clauses C1-C4, further comprising simultaneously subjecting the sample to multiplex amplification prior to the moving step.
C6. The method of any one of clauses C1-C5, wherein all steps are performed within a single closed system.
D1. A system for detecting which of a plurality of target nucleic acids is in a sample, the system comprising:
a vessel comprising a plurality of sample wells, each sample well configured for holding a portion of the sample, nucleic acid primers specific for amplification of one target nucleic acid, a fluorescent dye that produces a rising fluorescence signal in response to a rise a concentration of nucleic acid in the plurality of sample wells, and components for amplification, and
an instrument configured to concurrently subject the portion of the sample in each of the plurality of sample wells to amplification conditions and then melting conditions, the instrument comprising a detector for detecting a fluorescent signal from the fluorescent dye indicating amplification,
wherein the instrument is programmed with the target nucleic acids to be amplified in each one of the plurality of wells, melt range information for each of the target nucleic acids, and wherein the instrument is programmed to monitor fluorescence in the plurality of wells during the amplification conditions and, if during the amplification conditions, a sample well shows an increase in fluorescence indicative of positive nucleic acid amplification, then performing a melt detection, wherein the melt detection is defined by the melt temperature range for the target nucleic acid amplified in the sample well.
D2. The system of clause D1, wherein the instrument is programmed to perform a set number of amplification cycles prior to performing a melt detection.
D3. The system of clause D1 or D2, wherein the instrument is programmed to perform a melt detection at any amplification cycle number if amplification is detected in a sample well, and wherein a temperature range for the melt detection is limited by the melt range information for the target nucleic acid amplified in the well.
D4. The system of one of clauses D1-D3, wherein positive nucleic acid amplification is indicated by the fluorescence signal in the sample well rising above a threshold.
D5. The system of one of clauses D1-D4, wherein the threshold is one of a crossing point (Cp), a Cp plus one or more additional cycles of amplification, Ct, relative florescence units rising above a threshold value, mathematical modeling, signal processing, and combinations thereof.
D6. The system of any one of clause D1-D5, wherein the instrument is further programmed to not perform a melt detection if no sample well shows positive nucleic acid amplification within a set number of amplification cycles.
D7. The system of any one of clauses D1-D6, wherein the system is programmed to include a melt detection that comprises:
a first ramp rate during a first portion of the melt,
a second ramp rate during a second portion of the melt, and
a third ramp rate during a third portion of the melt,
the second ramp rate being slower than the first and third ramp rates and wherein the second portion of the melt is defined by the melt range information for the target nucleic acid amplified in the well.
D8. The system of any one of clauses D1-D7, wherein the system is programmed to identify at least one of a target organism, cell-free DNA, a cell, or a molecular marker for anti-microbial resistance present in the sample by identifying at least one corresponding sample well in which amplification and a melt detection have occurred.
D9. The system of any one of clauses D1-D8, wherein the amplification is PCR and the components include a polymerase and dNTPs.
D10. The system of any one of clauses D1-D9, wherein the instrument comprises a light source configured to emit optical signal toward the plurality of sample wells during screening of a respective sample.
D11. The system of any one of clauses D1-D10, wherein the melting result is based on the presence or absence of a melt peak within a predetermined temperature range, and wherein the presence of the melt peak outputs a positive result and the absence of the melt peak outputs a negative result.
E1. A system for detecting which of a plurality of target nucleic acids is in a sample, the system comprising:
a vessel comprising a plurality of sample wells, each sample well configured for holding a portion of the sample, nucleic acid primers specific for amplification of one target nucleic acid, a fluorescent dye that produces a rising fluorescence signal in response to a rise in concentration of nucleic acid in the plurality of sample wells, and components for amplification,
an instrument configured to concurrently subject the portion of the sample in each of the plurality of sample wells to amplification conditions and then melting conditions, the instrument comprising a detector for detecting a fluorescent signal from the fluorescent dye indicating amplification, and
programming for performing the methods of any one of clauses A1-C6,
wherein the instrument is programmed with the identity of the target nucleic acids to be amplified in each one of the plurality of wells and with melt range information for each of the target nucleic acids.
F1. A computer-implemented method for calling a sample for a target nucleic acid sequence, the method comprising:
sending, by one or more processors, control signals to a thermocycling element to heat a plurality of sample wells to a first temperature using an initial ramp rate and to cool the plurality of sample wells to a second temperature through one or more cycles, wherein each cycle includes an in-cycle temperature adjusting segment, the plurality of sample wells are each configured to house a portion of the sample, each sample well comprises the target nucleic acid sequence from a different one of a plurality of target nucleic acids, and each target nucleic acid from each different organism has a characteristic melt temperature range;
receiving, at the one or more processors from an optical system, data indicative of amounts of fluorescence emitted by the portions of the sample in the plurality of sample wells during the in-cycle temperature adjusting segment for the one or more cycles;
in response to determining that an amount of fluorescence for at least one of the sample wells exceeds a threshold:
   determining, by the one or more processors, the target nucleic acid sequence amplified in each of the sample wells exceeding the threshold;
   determining, by the one or more processors, an adjusted ramp rate profile for heating the sample well based on the melt temperature range and/or melt ramp rate characteristic for the target nucleic acid sequence in each of the sample wells exceeding the threshold; and
   performing, by the one or more processors, a melt detection for the portion of the sample including sending control signals to the thermocycling element to heat the sample well to the first temperature using the adjusted ramp rate profile for a subsequent cycle.
F2. The method of clause F1, wherein the first temperature is a denaturation temperature, the second temperature is an annealing temperature, the characteristic melt temperature range for the sample well is from an initial melt temperature to a final melt temperature, and the adjusted ramp rate profile includes:
a first ramp rate during an approach temperature range from the annealing temperature to the initial melt temperature;
a second ramp rate during the characteristic melt temperature range from the initial melt temperature to the final melt temperature; and
a third ramp rate during a finish temperature range from the final melt temperature to the denaturation temperature,
wherein the second ramp rate is slower than the first and third ramp rates.
F3. The method of clause F1 or clause F2, wherein the third ramp rate is faster than the first ramp rate.
F4. The method of any one of clauses F1-F3, wherein the second ramp rate is in a range of 0.05 to 4° C./sec.
F5. The method of any one of clauses F1-F4, wherein the first ramp rate is greater than 4° C./sec (e.g., between 6 and 20° C./sec), the second ramp rate is less than 4° C./sec (e.g., in a range of 0.05 to 4° C./sec), and the third ramp rate is greater than 4° C./sec (e.g., 12-20° C./sec).
F5.1. The method of any one of clauses F1-F5, wherein the third ramp rate is faster than the first ramp rate.
F5.2. The method of any one of clauses F1-F5.1, wherein the second ramp rate is in a range of 0.01 to 4° C./sec, or, preferably, 0.01 to 2° C./sec.
F5.3. The method of any one of clauses F1-F5.2, wherein the second ramp rate is preferably no greater than 0.5° C./sec, more preferably no greater than 1° C./sec, and most preferably no greater than 2° C./sec.
F5.4. The method of any one of clauses F1-F5.3, wherein the second ramp rate is adapted to detect a nucleic acid melting signature indicative of a genotype, a sequence variant, or a genetic mutation in a target nucleic acid that alters one or more of a nucleic acid melt temperature or a shape of a nucleic acid melting curve relative to the target nucleic acid without the genotype, sequence variant, or genetic mutation.
F5.5. The method of any one of clauses F1-F5.4, further comprising detecting a single nucleotide polymorphism (SNP).
F5.6. The method of any one of clauses F1-F5.5, further comprising detecting an antimicrobial resistance (AMR) marker.
F5.7. The method of any one of clauses F1-F5.6, further comprising detecting presence of an organism via a first melt detection in first assay and, if the organism is detected as present in the first assay, performing a second melt detection in a second assay to detect presence of or absence of the genotype, sequence variant, or genetic mutation.
F6. The method of any one of clauses F1-F5.7, wherein the threshold is one of a crossing point (Cp), a Cp plus one or more additional cycles of amplification, Ct, relative florescence units rising above a threshold value, mathematical modeling, signal processing, and combinations thereof.
F7. The method of any one of clauses F1-F6, wherein the controller is further configured to:
identify the target nucleic acid of the plurality of target nucleic acids corresponding to the sample well based on the melt detection.

F8. The method of any one of clauses F1-F7, wherein the controller is further configured to:
determine that the amount of fluorescence for a first sample well and a second sample well exceeds the threshold;
perform a first melt detection by using a first adjusted ramp rate profile corresponding to a first characteristic melt temperature range for melting the target nucleic acid amplified in the first sample well; and
perform a second melt detection by using a second adjusted ramp rate profile corresponding to a second characteristic melt temperature range for melting the target nucleic acid amplified in the second sample well.
G1. A system for calling a sample for a target nucleic acid sequence, the system comprising:
a plurality of sample wells each configured to house a portion of the sample, each sample well comprising the target nucleic acid sequence from a different one of a plurality of target nucleic acids, wherein each target nucleic acid from each different organism has a characteristic melt temperature range;
an optical system configured to detect an amount of fluorescence emitted by the sample;
a controller configured to:
   send control signals to a thermocycling element to heat the plurality of sample wells to a first temperature using an initial ramp rate and to cool the plurality of sample wells to a second temperature through one or more cycles, wherein each cycle includes an in-cycle temperature adjusting segment;
   receive data indicative of amounts of fluorescence emitted by the portions of the sample in the plurality of sample wells from the optical system during the in-cycle temperature adjusting segment for the one or more cycles;
   in response to determining that the amount of fluorescence for at least one of the sample wells exceeds a threshold:
      determine an adjusted ramp rate profile for heating the sample well based on the characteristic melt temperature range for the target nucleic acid sequence in the sample well exceeding the threshold; and
      perform a melt detection for the portion of the sample by sending control signals to the thermocycling element to heat the sample well to the first temperature using the adjusted ramp rate profile for a subsequent cycle.
G2. The system of clause G1, wherein the first temperature is a denaturation temperature, the second temperature is an annealing temperature, the characteristic melt temperature range for the sample well is from an initial melt temperature to a final melt temperature, and the adjusted ramp rate profile includes:
a first ramp rate during an approach temperature range from the annealing temperature to the initial melt temperature;
a second ramp rate during the characteristic melt temperature range from the initial melt temperature to the final melt temperature; and
a third ramp rate during a finish temperature range from the final melt temperature to the denaturation temperature,
wherein the second ramp rate is slower than the first and third ramp rates.
G3. The system of clause G1 or clause G2, wherein the third ramp rate is faster than the first ramp rate.
G4. The system of any one of clauses G1-G3, wherein the second ramp rate is the same as the initial ramp rate.
G5. The system of any one of clauses G1-G4, wherein the first ramp rate is greater than 4° C./sec (e.g., 6-20° C./sec), the second ramp rate is less than 4° C./sec (e.g., in a range of 0.01-4° C./sec or 1-2° C./sec), and the third ramp rate is greater than 4° C./sec (e.g., 6-20° C./sec).
G6. The system of any one of clauses G1-G5, wherein the threshold is one of a crossing point (Cp), a Cp plus one or more additional cycles of amplification, Ct, relative florescence units rising above a threshold value, mathematical modeling, signal processing, and combinations thereof.
G7. The system of any one of clauses G1-G6, wherein the controller is further configured to:
identify the target nucleic acid of the plurality of target nucleic acids corresponding to the sample well based on the melt detection.
G8. The system of any one of clauses G1-G7, wherein the controller is further configured to:
determine that the amount of fluorescence for a first sample well and a second sample well exceeds the threshold;
perform a first melt detection by using a first adjusted ramp rate profile corresponding to a first characteristic melt temperature range for melting the target nucleic acid amplified in the first sample well; and
perform a second melt detection by using a second adjusted ramp rate profile corresponding to a second characteristic melt temperature range for melting the target nucleic acid amplified in the second sample well.
H1. A computing device for calling a sample for a target nucleic acid sequence comprising:
one or more processors; and
a non-transitory computer-readable memory coupled to the one or more processors and storing thereon instructions that, when executed by the one or more processors, cause the computing device to:
   send control signals to a thermocycling element to heat a plurality of sample wells to a first temperature using an initial ramp rate and to cool the plurality of sample wells to a second temperature through one or more cycles, wherein each cycle includes an in-cycle temperature adjusting segment, the plurality of sample wells are each configured to house a portion of the sample, each sample well comprises the target nucleic acid sequence from a different one of a plurality of target nucleic acids, and each target nucleic acid from each different organism has a characteristic melt temperature range;
   receive, from an optical system, data indicative of amounts of fluorescence emitted by the portions of the sample in the plurality of sample wells during the in-cycle temperature adjusting segment for the one or more cycles;
   in response to determining that the amount of fluorescence for at least one of the sample wells exceeds a threshold:
      determine an adjusted ramp rate profile for heating the sample well based on the characteristic melt temperature range for the target nucleic acid sequence in the sample well exceeding the threshold; and
      perform a melt detection for the portion of the sample by sending control signals to the thermocycling element to heat the sample well to the first temperature using the adjusted ramp rate profile for a subsequent cycle.
H2. The computing device of clause H1, wherein the first temperature is a denaturation temperature, the second temperature is an annealing temperature, the characteristic melt temperature range for the sample well is from an initial melt temperature to a final melt temperature, and the adjusted ramp rate profile includes:
a first ramp rate during an approach temperature range from the annealing temperature to the initial melt temperature;
a second ramp rate during the characteristic melt temperature range from the initial melt temperature to the final melt temperature; and
a third ramp rate during a finish temperature range from the final melt temperature to the denaturation temperature,
wherein the second ramp rate is slower than the first and third ramp rates.
H3. The computing device of clause H1 or clause H2, wherein the third ramp rate is faster than the first ramp rate.
H4. The computing device of any one of clauses H1-H3, wherein the second ramp rate is the same as the initial ramp rate.
H5. The computing device of any one of clauses H1-H4, wherein the first ramp rate is greater than 4° C./sec (e.g., 6-20° C./sec), the second ramp rate is less than 4° C./sec (e.g., in a range of 0.01-4° C./sec or 1-2° C./sec), and the third ramp rate is greater than 4° C./sec (e.g., 6-20° C./sec).
H6. The computing device of any one of clauses H1-H5, wherein the threshold is one of a crossing point (Cp), a Cp plus one or more additional cycles of amplification, Ct, relative florescence units rising above a threshold value, mathematical modeling, signal processing, and combinations thereof.
H7. The computing device of any one of clauses H1-H6, wherein the instructions further cause the computing device to:
identify the target nucleic acid of the plurality of target nucleic acids corresponding to the sample well based on the melt detection.
H8. The computing device of any one of clauses H1-H7, wherein the instructions further cause the computing device to:
determine that the amount of fluorescence for a first sample well and a second sample well exceeds the threshold;
perform a first melt detection by using a first adjusted ramp rate profile corresponding to a first characteristic melt temperature range for melting the target nucleic acid amplified in the first sample well; and
perform a second melt detection by using a second adjusted ramp rate profile corresponding to a second characteristic melt temperature range for melting the target nucleic acid amplified in the second sample well.

Reaction vessels and devices that suitably may be used with any of the methods described herein are also provided in this disclosure.

Additional features of the present invention will become apparent to those skilled in the art upon consideration of the following detailed description of preferred embodiments exemplifying the best mode of carrying out the invention as presently perceived.

### BRIEF DESCRIPTION OF THE FIGURES

FIG. 1 shows an illustrative pouch that suitably may be used in embodiments of this disclosure.

FIG. 2 shows illustrative stages of a melting procedure that suitably may be used in embodiments of this disclosure.

FIGS. 3A and 3B show examples comparing a slow melt with a fixed temperature ramp rate to fast melts with a fixed temperature ramp rate.

FIG. 4A shows an example comparing a slow melt with a fixed ramp rate to a dynamic melt with a variable ramp rate using faster ramp rates at temperature ranges where no melt signature is expected from the reaction, and a slower ramp rate at a temperature range where a melt signature is expected from the reaction.

FIG. 4B shows another example similar to FIG. 4A comparing a slow melt with a fixed ramp rate to a dynamic melt with a variable ramp rate.

FIG. 5A illustrates an example of a dynamic melting analysis for one assay target.

FIG. 5B shows an example of dynamic melting after a fixed number of cycles of amplification with the target of FIG. 5A.

FIG. 6A illustrates an example of a dynamic melting analysis for two assay targets with one melt temperature range.

FIG. 6B shows an example of dynamic melting after a fixed number of cycles of amplification with the two targets of FIG. 6A.

FIG. 7A shows an example of dynamic melting analysis with one analyte after a number of cycles determined dynamically instead of after a fixed number of cycles of amplification.

FIG. 7B shows the dynamic melting example of FIG. 7A with the melt being performed based on when the analyte showed amplification above a threshold value.

FIG. 8A illustrates an example of assay specific melt detection with one dynamic melt for each target and the number of cycles of amplification prior to each melt being determined dynamically based on when each target showed amplification above a threshold value.

FIG. 8B illustrates melt specific parameters for the first target of FIG. 8A.

FIG. 8C illustrates the results of the dynamic melt for the first target illustrated in FIGS. 8A and 8B.

FIG. 8D illustrates melt specific parameters for the second target of FIG. 8A.

FIG. 8E illustrates the results of the dynamic melt for the second target illustrated in FIGS. 8A and 8D.

FIG. 9 illustrates a block diagram of an exemplary embodiment of a thermal cycling system in accordance with aspects of the disclosure.

FIG. 10 illustrates a flow diagram of an example computer-implemented dynamic melt detection method for calling a sample for a target nucleic acid sequence.

FIG. 11A illustrates an example of a temperature ramping profile for a nucleic acid melting experiment.

FIG. 11B illustrates an example of nucleic acid melting curves (left panel) and derivative melt curves (right panel) for two amplicons with different melting temperatures.

FIG. 12 illustrates schematic melt procedures that compare a standard melt protocol ( [symbol] ), a dynamic melt protocol ( [symbol] ), and a dynamic “high resolution” protocol ( [symbol] [symbol] ).

FIG. 13 illustrates an N. gonorrhoeae organism assay (● Org) and a separate assay targeted to a mutation prone region of the gyrA gene (∘ S91F and +WT).

FIGS. 14A and 14B compare melt curves for wild-type ( [symbol] MG-WT) and A2059G ( [symbol] 2059G) mutant amplicons of the 23S ribosomal RNA gene of M. genitalium.

FIGS. 15A and 15B compare melt curves for wild-type ( [symbol] MG-WT) and A2058G ( [symbol] 2058G) mutant amplicons of the 23S ribosomal RNA gene of M. genitalium.

FIGS. 16A and 16B compare melt curves for wild-type ( [symbol] NG-WT) and S91F ( [symbol] S91F) mutant amplicons of the gyrA gene of N. gonorrhoeae.

### DETAILED DESCRIPTION

As used herein, the terms “a,” “an,” and “the” are defined to mean one or more and include the plural unless the context is inappropriate. Ranges may be expressed herein as from “about” one particular value, and/or to “about” another particular value. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 5%. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

The word “or” as used herein means any one member of a particular list and also includes any combination of members of that list.

By “sample” is meant an animal; a tissue or organ from an animal; a cell (either within a subject, taken directly from a subject, or a cell maintained in culture or from a cultured cell line); a cell lysate (or lysate fraction) or cell extract; a solution containing one or more molecules derived from a cell, cellular material, or viral material (e.g. a polypeptide or nucleic acid); or a solution containing a non-naturally occurring nucleic acid, which is assayed as described herein. A sample may also be any body fluid or excretion (for example, but not limited to, blood, urine, stool, saliva, tears, bile, cerebrospinal fluid) that contains cells, cell components, or nucleic acids.

As used herein, the term “reaction” may refer to nucleic acid amplification reaction (e.g., a PCR reaction) that is used to amplify a given target nucleic acid. As used herein, the term “assay” may be used to refer to one or more reactions in a single well or container. As used herein, the term “panel” may be used to refer to a collection of assays that may be grouped together to test for a group of targets (e.g., a panel to test for respiratory pathogens). For example, a panel may be designed to include a number of assays to amplify and detect 20 or more targets.

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids of the invention can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof

By “probe,” “primer,” or “oligonucleotide” is meant a single-stranded DNA or RNA molecule of defined sequence that can base-pair to a second DNA or RNA molecule that contains a complementary sequence (the “target”). The stability of the resulting hybrid depends upon the length, GC content, and the extent of the base-pairing that occurs. The extent of base-pairing is affected by parameters such as the degree of complementarity between the probe and target molecules and the degree of stringency of the hybridization conditions. The degree of hybridization stringency is affected by parameters such as temperature, salt concentration, and the concentration of organic molecules such as formamide, and is determined by methods known to one skilled in the art. Probes, primers, and oligonucleotides may be detectably-labeled, either radioactively, fluorescently, or non-radioactively, by methods well-known to those skilled in the art. dsDNA binding dyes may be used to detect dsDNA. It is understood that a “primer” is specifically configured to be extended by a polymerase, whereas a “probe” or “oligonucleotide” may or may not be so configured.

By “dsDNA binding dyes” is meant dyes that fluoresce differentially when bound to double-stranded DNA than when bound to single-stranded DNA or free in solution, usually by fluorescing more strongly. While reference is made to dsDNA binding dyes, it is understood that any suitable dye may be used herein, with some non-limiting illustrative dyes described in U.S. Pat. No. 7,387,887, herein incorporated by reference. Other signal producing substances may be used for detecting nucleic acid amplification and melting, illustratively enzymes, antibodies, etc., as are known in the art.

By “specifically hybridizes” is meant that a probe, primer, or oligonucleotide recognizes and physically interacts (that is, base-pairs) with a substantially complementary nucleic acid (for example, a sample nucleic acid) under high stringency conditions, and does not substantially base pair with other nucleic acids.

By “high stringency conditions” is meant conditions that allow hybridization comparable with that resulting from the use of a DNA probe of at least 40 nucleotides in length, in a buffer containing 0.5 M NaHPO₄, pH 7.2, 7% SDS, 1 mM EDTA, and 1% BSA (Fraction V), at a temperature of 65° C., or a buffer containing 48% formamide, 4.8×SSC, 0.2 M Tris-Cl, pH 7.6, 1×Denhardt's solution, 10% dextran sulfate, and 0.1% SDS, at a temperature of 42° C. Other conditions for high stringency hybridization, such as for PCR, Northern, Southern, or in situ hybridization, DNA sequencing, etc., are well known by those skilled in the art of molecular biology.

While PCR is the amplification method used in the examples herein, it is understood that any amplification method that uses a primer may be suitable. Such suitable procedures include polymerase chain reaction (PCR); strand displacement amplification (SDA); nucleic acid sequence-based amplification (NASBA); cascade rolling circle amplification (CRCA), loop-mediated isothermal amplification of DNA (LAMP); isothermal and chimeric primer-initiated amplification of nucleic acids (ICAN); target based-helicase dependent amplification (HDA); transcription-mediated amplification (TMA), and the like. Therefore, when the term PCR is used, it should be understood to include other alternative amplification methods. For amplification methods without discrete cycles, reaction time may be used where measurements are made in cycles or Cp, and additional reaction time may be added where additional PCR cycles are added in the embodiments described herein. It is understood that protocols may need to be adjusted accordingly.

Various embodiments disclosed herein use a self-contained nucleic acid analysis pouch to interrogate a sample for the presence of various biological substances, illustratively antigens and nucleic acid sequences, illustratively in a single closed system. Such systems, including pouches and instruments for use with the pouches, are disclosed in more detail in U.S. Pat. Nos. 8,394,608; 8,895,295; and 10,464,060, herein incorporated by reference. However, it is understood that such pouches are illustrative only, and the multiple PCR reactions discussed herein may be performed in any of a variety of open or closed system sample vessels as are known in the art, including 96-well plates, plates of other configurations, arrays, carousels, and the like, using a variety of amplification systems, as are known in the art. While the term “sample well” is used herein, this term is meant to encompass wells, tubes, and various other reaction containers, as are used in these amplification systems. In one embodiment, the pouch is used to assay for multiple pathogens. Illustratively, various steps may be performed in the optionally disposable pouch, including nucleic acid preparation, primary large volume multiplex PCR, dilution of primary amplification product, and secondary PCR, culminating with optional real-time detection or post-amplification analysis such as melting-curve analysis. Further, it is understood that while the various steps may be performed in pouches of the present invention, one or more of the steps may be omitted for certain uses, and the pouch configuration may be altered accordingly.

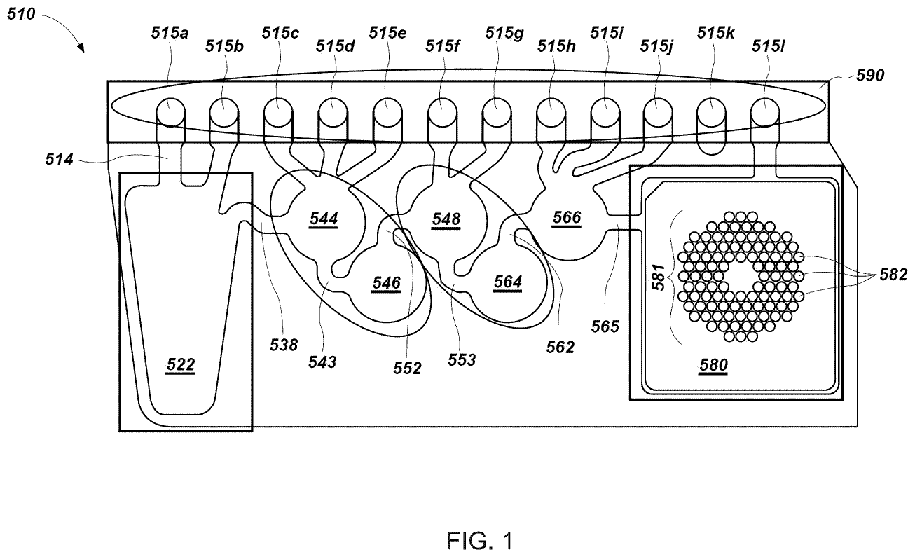

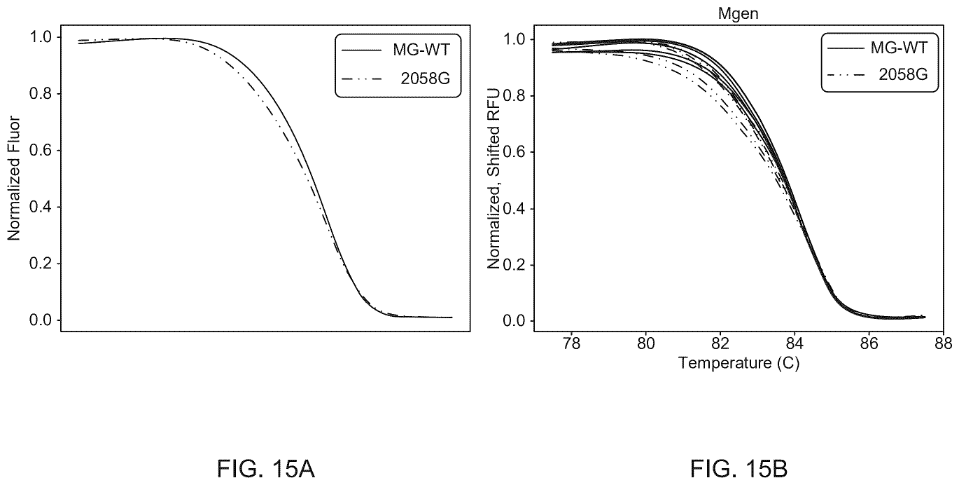

FIG. 1 shows an illustrative pouch 510 for use with the current invention. Pouch 510 is similar to FIG. 15 of U.S. Pat. No. 8,895,295, already incorporated by reference, with like items numbered the same. Fitment 590 is provided with entry channels 515a through 5151, which also serve as reagent reservoirs. Illustratively, reagents may be freeze dried in fitment 590 and rehydrated prior to use. Blisters 522, 544, 546, 548, 564, and 566, with their respective channels 538, 543, 552, 553, 562, and 565 are similar to blisters of the same number of FIG. 15 of U.S. Pat. No. 8,895,295. Second-stage reaction zone 580 of FIG. 1 is similar to that of U.S. Pat. No. 8,895,295, but the second-stage wells 582 of high-density array 581 are arranged in a somewhat different pattern. The more circular pattern of high-density array 581 of FIG. 1 eliminates corners and can result in more uniform filling of second-stage wells 582. As shown, the high-density array 581 is provided with 102 second-stage wells 582. Pouch 510 is suitable for use in the FilmArray instrument. However, it is understood that the pouch embodiment is illustrative only.

Pouch 510 may be used in a manner similar to that described in U.S. Pat. No. 8,895,295, already incorporated by reference. A 300 μl mixture comprising the sample to be tested (100 μl) and lysis buffer (200 μl) is injected into injection port (not shown) in fitment 590 near entry channel 515a, and the sample mixture is drawn into entry channel 515a. Water is also injected into a second injection port (not shown) of the fitment 590 adjacent entry channel 515l, and is distributed via a channel (not shown) provided in fitment 590, thereby hydrating up to eleven different reagents, each of which were previously provided in dry form at entry channels 515b through 5151 These reagents illustratively may include freeze-dried PCR reagents, DNA extraction reagents, wash solutions, immunoassay reagents, or other chemical entities. Illustratively, the reagents are for nucleic acid extraction, first-stage multiplex PCR, dilution of the multiplex reaction, and preparation of second-stage PCR reagents, as well as control reactions. In the embodiment shown in FIG. 1, all that need be injected is the sample solution in one injection port and water in the other injection port. After injection, the two injection ports may be sealed. For more information on various configurations of pouch 510 and fitment 590, see U.S. Pat. No. 8,895,295, already incorporated by reference.

After injection, the sample is moved from injection channel 515a to lysis blister 522 via channel 514. Lysis blister 522 is provided with ceramic beads and is configured for vortexing via impaction using rotating blades or paddles provided within the FilmArray instrument. Once the cells have been adequately lysed, the sample is moved through channel 538, blister 544, and channel 543, to blister 546, where the sample is mixed with nucleic acid-binding magnetic beads. The mixture is allowed to incubate for an appropriate length of time, illustratively approximately 10 seconds to 10 minutes. A retractable magnet located within the FilmArray instrument adjacent blister 546 captures the magnetic beads from the solution, forming a pellet against the interior surface of blister 546. The liquid is then moved out of blister 546 and back through blister 544 and into blister 522, which is now used as a waste receptacle. One or more wash buffers from one or more of injection channels 515c to 515e are provided via blister 544 and channel 543 to blister 546. Optionally, the magnet is retracted and the magnetic beads are washed by moving the beads back and forth from blisters 544 and 546 via channel 543. Once the magnetic beads are washed, the magnetic beads are recaptured in blister 546 by activation of the magnet, and the wash solution is then moved to blister 522. This process may be repeated as necessary to wash the lysis buffer and sample debris from the nucleic acid-binding magnetic beads.

After washing, elution buffer stored at injection channel 515f is moved to blister 548, and the magnet is retracted. The solution is cycled between blisters 546 and 548 via channel 552, breaking up the pellet of magnetic beads in blister 546 and allowing the captured nucleic acids to dissociate from the beads and come into solution. The magnet is once again activated, capturing the magnetic beads in blister 546, and the eluted nucleic acid solution is moved into blister 548.

First-stage PCR master mix from injection channel 515g is mixed with the nucleic acid sample in blister 548. Optionally, the mixture is mixed by forcing the mixture between 548 and 564 via channel 553. After several cycles of mixing, the solution is contained in blister 564, where a pellet of first-stage PCR primers is provided, at least one set of primers for each target organism, and first-stage multiplex PCR is performed. If RNA targets are present, an RT step may be performed prior to or simultaneously with the first-stage multiplex PCR. First-stage multiplex PCR temperature cycling in the FilmArray instrument is illustratively performed for 15-20 cycles, although other levels of amplification may be desirable, depending on the requirements of the specific application.

After first-stage PCR has proceeded for the desired number of cycles, the sample may be diluted, illustratively by forcing most of the sample back into blister 548, leaving only a small amount in blister 564, and adding second-stage PCR master mix from injection channel 515i. Alternatively, a dilution buffer from 515i may be moved to blister 566 then mixed with the amplified sample in blister 564 by moving the fluids back and forth between blisters 564 and 566. If desired, dilution may be repeated several times, using dilution buffer from injection channels 515j and 515k, and then adding second-stage PCR master mix from injection channel 515h to some or all of the diluted amplified sample. It is understood that the level of dilution may be adjusted by altering the number of dilution steps or by altering the percentage of the sample discarded prior to mixing with the dilution buffer or second-stage PCR master mix comprising components for amplification, illustratively a polymerase, dNTPs, and a suitable buffer, although other components may be suitable, particularly for non-PCR amplification methods. If desired, this mixture of the sample and second-stage PCR master mix may be pre-heated in blister 564 prior to movement to second-stage wells 582 for second-stage amplification. Such preheating may obviate the need for a hot-start component (antibody, chemical, or otherwise) in the second-stage PCR mixture.

The illustrative second-stage PCR master mix is incomplete, lacking primer pairs, and each of the 102 second-stage wells 582 is pre-loaded with a specific PCR primer pair. If desired, second-stage PCR master mix may lack other reaction components, and these components may be pre-loaded in the second-stage wells 582 as well. Each primer pair may be similar to or identical to a first-stage PCR primer pair or may be nested within the first-stage primer pair. Movement of the sample from blister 564 to the second-stage wells 582 completes the PCR reaction mixture. Once high-density array 581 is filled, the individual second-stage reactions are sealed in their respective second-stage blisters by any number of means, as is known in the art. Illustrative ways of filling and sealing the high-density array 581 without cross-contamination are discussed in U.S. Pat. No. 8,895,295, already incorporated by reference. Illustratively, the various reactions in wells 582 of high-density array 581 are simultaneously thermal cycled, illustratively with one or more peltier devices, although other means for thermal cycling are known in the art.

The illustrative second-stage PCR master mix contains the dsDNA binding dye LCGreen® Plus to generate a signal indicative of amplification. However, it is understood that this dye is illustrative only, and that other signals may be used, including other dsDNA binding dyes, and probes that are labeled fluorescently, radioactively, chemiluminescently, enzymatically, or the like, as are known in the art.

The illustrative FilmArray instrument is programmed to make positive or negative calls for each second-stage reaction based on a post-PCR melt. The melt curve must produce a melt peak (first derivative maximum or negative first derivative maximum) within a pre-defined temperature range, for the call to be positive. It is understood that this method of calling each second-stage reaction is illustrative only, and that calls could be made using real-time amplification data or by other means, as are known in the art.

### Example 1

Amplicon melting at the conclusion of nucleic acid amplification (e.g., PCR) is designed to confirm that the amplification product is, in fact, the intended or desired product. That is, real-time monitoring of nucleic acid amplification can be used to identify the presence of amplification, but it does not tell the user what was amplified. The amplification product could be the intended product or a product of contamination, the result of non-specific amplification, etc. Because amplicons may be designed to have specific and known melt characteristics, post-amplification melting can be used to confirm that the correct product was made.

Many nucleic acid amplification reactions include a nucleic acid binding dye that is capable of incorporating into double stranded nucleic acid (e.g., dsDNA). The dye is incorporated into the double stranded nucleic acid as it is produced in the reaction. Many nucleic acid binding dyes are known to be compatible with nucleic acid amplification conditions, bind tightly to double-stranded nucleic acid, and fluoresce strongly in the presence of double-stranded nucleic acid. When the nucleic acid is denatured (e.g., by high temperature), the dye is released and the dye fluoresces weakly or not at all in the presence of single stranded nucleic acid. Such nucleic acid binding dyes also typically fluoresce in the presence of double stranded nucleic acid in a concentration dependent fashion—i.e., more double stranded nucleic acid in a reaction produces greater fluorescence. This can be used to monitor the success (or failure) of a nucleic acid amplification reaction in real time by watching for an increase in fluorescence above a threshold value. Examples of a “threshold value” used in the embodiments and claims described herein to determine when fluorescence has increased and a melt can be performed include, but are not limited to, one of a crossing point (Cp), a Cp plus one or more additional cycles of amplification, a crossing threshold (Ct), relative florescence units rising above a threshold value, relative florescence units at a selected value above a threshold value (e.g., 2 RFUs, 5 RFUs, 10 RFUs, etc.), mathematical modeling, signal processing, and combinations thereof.

As used herein “Cp” or “crossing point” means the number of cycles or fractional cycles of PCR required to obtain a fluorescence signal above a predetermined threshold value. For instance, the threshold value may be the level of detection or the point at which a measure of fluorescence for a reaction reaches an intensity above a background level of fluorescence. Cp may be determined experimentally based on a manually set threshold, although other methods for determining Cp are known in the art. Other points may be used as well, such as using a first, second, or nth order derivative, illustratively as taught in U.S. Pat. No. 6,303,305, herein incorporated by reference in its entirety. Other points may be used as well, as are known in the art, and any such point may be substituted for Cp in any of the methods discussed herein. The terms “Ct,” “crossing threshold,” and “Cq” are generally synonymous with crossing point (Cp) and these terms may be used interchangeably.

“Relative fluorescence units” (RFU) are defined herein as fluorescence intensity values reported by reference to another value. Fluorescence units are a dimensionless term and, as such, the intensity of a fluorescent signal is usually reported relative to another measurement or to a reference measurement taken by an instrument. For example, a baseline fluorescence value may be measured and RFUs may then be reported as multiples of the baseline value—i.e., 2 RFUs, 5 RFUs, 10 RFUs, etc. Suitable examples of baseline values may include, but are not limited to, background fluorescence or any arbitrary value above or below baseline. In any given experiment or set of experiments using RFUs, the important thing is to use a consistent and repeatable value as baseline.

As used herein “Tm” is the temperature at which one-half of the DNA duplex will dissociate to become single stranded. In amplification systems, Tm is usually measured subsequent to amplification, although techniques are known for measuring Tm during amplification.

Below are two examples of methods of determining if amplification has occurred and if it is appropriate to trigger a melt detection. These and similar methods may be used with any of the methods and systems described and claimed herein.

In a first example, a set of reaction wells includes wells with all amplification reagents except for amplification primers (empty wells) and wells with all amplification reagents including amplification primers and nucleic acid template that can be amplified by the reaction (target wells). In a nonlimiting example, the method may treat the average fluorescence of the empty wells as baseline and subtract the baseline fluorescence from the target well florescence at a given cycle N (e.g., cycle 5). Other methods for determining baseline fluorescence are known in the art and may be used with the methods described herein. A target may be run in triplicate (i.e., the target may be amplified in three separate target wells). If the value (the difference between a target well at cycle N and the average of the empty wells at cycle N) is above a threshold value for at least one of the three target wells, at least two of the three target wells, or all three target wells, a melt may be triggered. The basis of the method of using the empty wells to define the background is checking to see if the RFU for the target wells has changed enough relative to baseline that it can be decided that amplification has occurred. This method may be used for assays that would be expected to amplify and show a detectable Cp within a number of cycles designed for the assay (e.g., within 15-30 cycles of PCR) if the target is present.

A second non-limiting example is designed for assays needing greater sensitivity. An algorithm similar to the previous example may be used, but, instead of comparing target wells to empty wells to detect whether or not amplification has occurred, at least one target well (e.g., three target wells) may be compared to itself at cycle N and at an earlier cycle (e.g., N−1, N−2, N−3, etc.). Target wells are still first “compared” to empty wells in order to subtract the empty well fluorescence (i.e., the background fluorescence). This is still important for normalization of global changes in fluorescence that would be seen in both target wells and empty wells. If the increase in the fluorescence value from the earlier cycle (e.g., N−1) to cycle Nis above a threshold value for at least one of the three target wells, at least two of the three target wells, or all three target wells, a melt may be triggered. The basis of this method is checking to see if the RFU is changing quickly enough that it can be decided that amplification is occurring. This algorithm may be used for assays where low level amplification may occur at the end of cycling (possibly without a typical Cp call) but a melt can still be called in response to amplification.

Copies of double-stranded nucleic acid generated during a nucleic acid amplification reaction (called products or amplicon) will have unique sequences based on the template that was amplified. Amplicon length and sequence/composition (typically, A-T/G-C content) determines the temperature at which the double-stranded DNA will melt apart, which is known as the melting temperature (Tm) of the amplicon. Products made from different targets will have different sequences and, therefore, different Tms. As such, the nucleic acid binding dye added to the reaction can also be used for nucleic acid melting analysis. A specific product will have a specific melting signature (i.e., a temperature transition range over which fluorescence decays as the duplex transitions from fully double stranded to fully single stranded), so nucleic melting can be used to confirm the presence of the correct product in a nucleic acid amplification reaction.

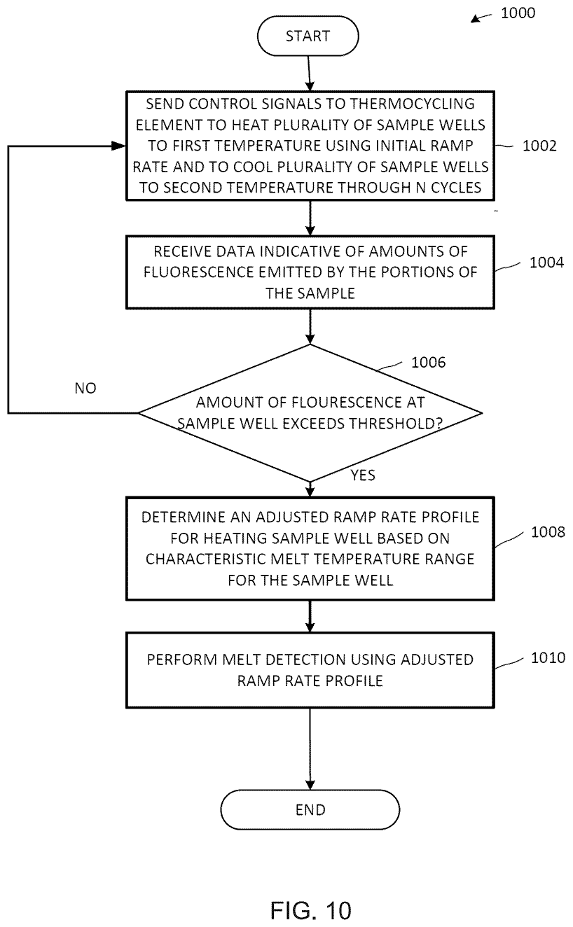

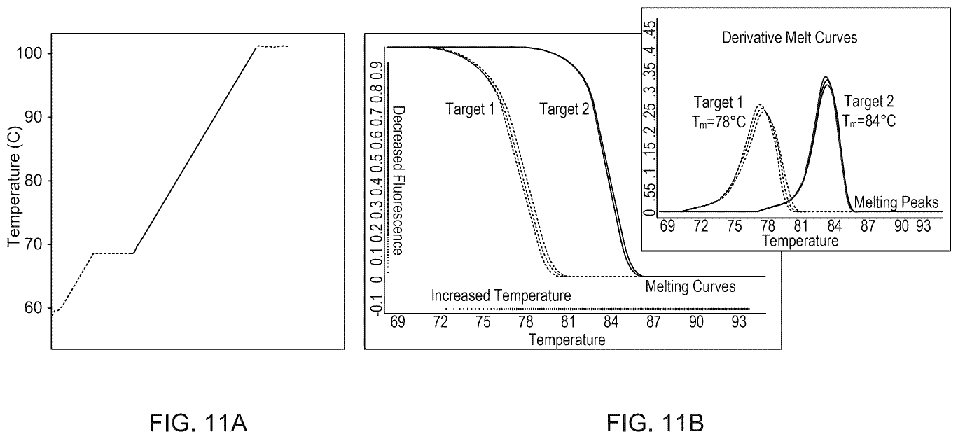

A typical melting experiment is performed by gradually raising the temperature of the reaction through a range starting at or below the annealing temperature to above the denaturation temperature (e.g., from approximately 60° C. to 98° C.). See, e.g., FIG. 11A. As the temperature reaches the Tm of an amplicon, the amplicon denatures and fluorescence drops as the nucleic acid binding dye is released. This produces a melting curve, seen in the left panel of FIG. 11B, which shows the rapid decline in fluorescence upon amplicon melting. A melting peak with a specific Tm, shown in the right panel of FIG. 11B, is generated for each amplicon by plotting the negative first derivative of the melting curve.

Melting analysis may be used to identify specific PCR product. Since the sequence and Tm of an amplicon from a specific target is known and consistent, specific PCR product can be identified as being copied from that target. Non-specific PCR products, contamination products, and the like with different Tms may be excluded.

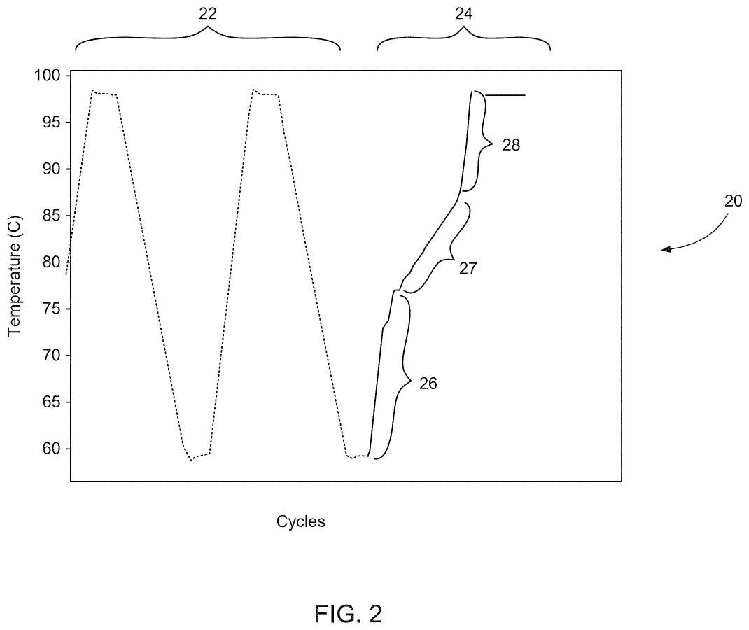

In contrast to a typical melt temperature ramp like the one shown in FIG. 11A, FIG. 2 shows illustrative stages of a melting procedure 20 that suitably may be used in embodiments of this disclosure. Melting procedure 20 illustratively shows the final two cycles 22 of nucleic acid amplification and then a melt temperature ramp 24. The cycles of nucleic acid amplification may come at the end of amplification, followed by the melt temperature ramp, or the melt temperature ramp may come at some interim stage of amplification (e.g., after 10-15 cycles of amplification) followed by additional cycles of amplification. Unlike the typical melt ramp shown in FIG. 11A, melt temperature ramp 24 includes different ramp rates 26, 27, and 28 between a low annealing temperature and a high denaturation temperature (e.g., between about 60° C. and about 98° C.). The different ramps rates 26, 27, and 28 suitably may be selected so that the instrument is ramping the temperature rapidly in areas (e.g., approach area 26 and finish area 28) of the melt temperature ramp 24 where no melt information is expected (i.e., the nucleic acid duplex is either an unmelted duplex (approach area 26) or is it fully melted (finish area 28)) and ramped more slowly in a region of interest (e.g., range 27) where the melt transition of the relevant amplicon (or amplicons) is expected (i.e., the transition from double-stranded to single-stranded). For example, the temperature in ranges 26 and 28 may be ramped rapidly (greater than 4° C./sec, e.g., at 4-20° C./sec in ranges 26 and 28) and temperature in range 27 may be ramped more slowly (less than 4° C./sec, e.g., 0.01-4° C./sec, or e.g., 1-2° C./sec). The melt ramp rates in the foregoing are illustrative and non-limiting to the disclosure herein. It is possible to ramp temperature up much faster than 20°/sec (e.g., 100° C./sec or greater) and some melts may be performed with ramp rates of less than 0.01° C./sec and greater than 4° C./sec. This novel melt procedure can save significant time during the melt analysis because the temperature can be ramped rapidly in portions of the melt cycle where no melting information is expected and can, in turn, be ramped slowly only for a specific window where the relevant amplicon or amplicons is/are expected to melt. This novel melt procedure can also preserve the high resolution of a slowly ramped melt because the temperature is ramped slowly in the narrow region of interest around the melt transition temperature for the selected amplicon. While the devices described herein may be designed to test for the presence of many potential targets (e.g., up to 30 or more organisms and/or molecular markers), a typical test case is likely to include either none or only one or two positives. The present invention targets the melt ramp to the amplicons that are actually present instead of ramping slowly through an entire melt window that is designed to encompass all possible amplicons.

The different ramps rates shown at 26, 27, and 28 suitably may be selected dynamically according to the invention described herein. Referring again to FIG. 1, the second stage wells 582 of second stage reaction zone 580 are each spotted with a specific primer pair for amplification of one target nucleic acid. Amplification for some targets suitably may be performed in duplicate or triplicate, so identical primer pairs may be spotted in more than one second stage well 582. The spotting of the second stage wells 582 is a specific part of assay design and is carefully mapped. As such, an instrument with instrument software designed to perform a test with pouch 510 knows the location of each second stage well 582 and, for a given assay, knows beforehand the identity of and details about the target amplicon(s) expected to be amplified in each second stage well 582. When a well in the second stage reaction zone 580 shows fluorescence indicative of successful amplification, the melt characteristics of the target amplicon in that well are typically known and the identity of the assay (e.g., an assay for detecting an organism in a sample) associated with that well is also known. This description of assay design and primer spotting is specific to FilmArray, but persons of ordinary skill will appreciate that the principle described can be applied to any test using nucleic acid amplification, such as, but not limited to, a tube-based or a 96-well plate-based test. As such, the rapidly ramped ranges 26 and 28 and the slowly ramped temperature range 27 suitably may be selected based on the expected melt temperature range of a specific amplicon in a sample well for which positive amplification has been detected. The beginning and end points of the slow ramp range 27 suitably may be selected dynamically from a number of amplicon melt ranges in an assay depending on which assay (i.e., which reactions in the sample well(s)) in the panel are actually positive for amplification.

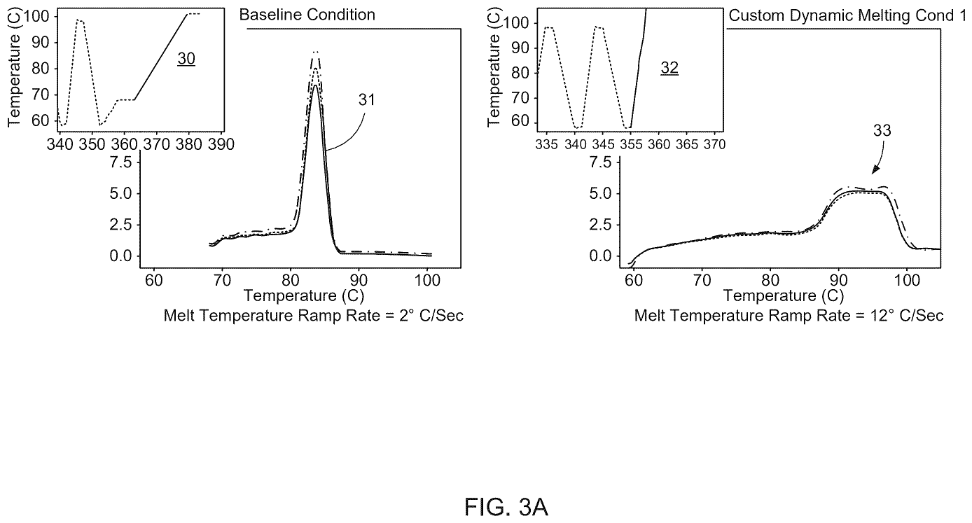

Referring now to FIGS. 3A and 3B, examples are shown comparing slow melts with fixed temperature ramp rates to fast melts with a more rapid fixed temperature ramp rate. The difference between FIGS. 3A and 3B is that the melts are based on amplification fluorescence at 10 RFU (FIG. 3A) versus 4 RFU (FIG. 3B). In other words, the amplicons in FIG. 3A are amplified to a higher concentration than those in FIG. 3B; this difference yields a stronger fluorescence signal in FIG. 3A relative to FIG. 3B. Negative first derivatives for the slow melts are shown at 31 of FIG. 3A and 35 of FIG. 3B. The negative first derivative curves shown at 31 and 35 are similar to the negative first derivative melt curves shown in FIG. 11B. For the negative first derivative curves shown at 31 and 35 shown in FIGS. 3A, 3B, and 11B, the temperatures were ramped at approximately 2° C./sec through a range starting at or below the annealing temperature to above the denaturation temperature (e.g., from approximately 60° C. to 98° C.). This is a standard melt procedure for FilmArray. In each case, the melt peaks shown in the negative derivative curves are clear and unambiguous. In contrast, FIGS. 3A and 3B show negative derivative curves 33 and 37 that were produced from the 12° C./sec temperature ramp rates shown at 32 and 36. The only difference between negative derivative curves 31 and 35 and negative derivative curves 33 and 37 is the temperature ramp rate. As can be seen from negative derivative curves 33 and 37, the peak between curves 31 and 33 is flattened and shifted to a higher temperature and the negative derivative curve 37 shows no peak compared to negative derivative curve 35. These fast melts illustrate that fast ramping through the entire melt range may not be adequate to retain melt signal compared to standard melts.

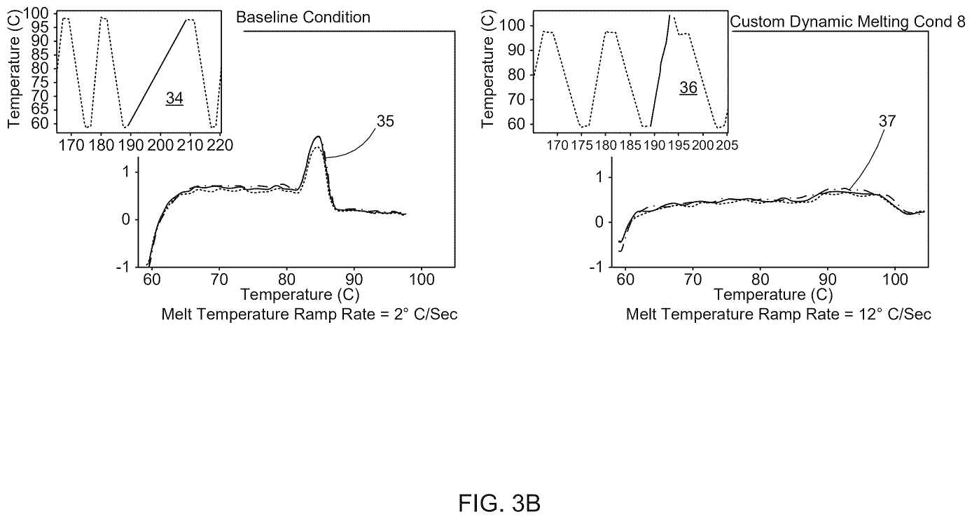

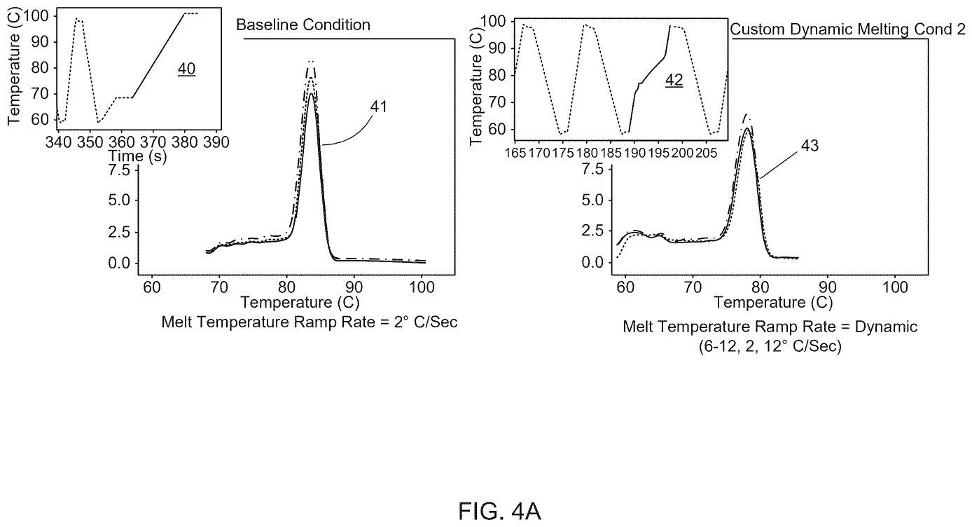

Referring now to FIGS. 4A and 4B, examples are shown comparing slow melts with a fixed ramp rate to melts with a variable ramp rate using faster ramp rates at temperature ranges where no melt signature is expected, and a slower ramp rate at a temperature range where a melt signature is expected (fast-slow-fast). The difference between FIGS. 4A and 4B is concentration of the nucleic acids at the time of the melt and, thus, the strength of the fluorescence signal: 10 RFU (FIG. 4A) versus 4 RFU (FIG. 4B). In other words, the amplicons in FIG. 4A were amplified to a higher concentration prior to melting analysis than those in FIG. 4B; this difference yields a stronger fluorescence signal in FIG. 4A relative to FIG. 4B. These relative RFUs are similar to the cases shown in FIGS. 3A and 3B. In spite of the differences in the threshold concentrations (i.e., 10 RFU vs. 4 RFU) prior to the melt analysis, the derivative melt curves for both sets of data are clear and unambiguous. The 2° C./sec melts shown in FIGS. 3A-4B are essentially identical. However, the melts using the modified dynamic (i.e., fast-slow-fast (6-12° C./sec, 2° C./sec, 12° C./sec)) melt ramping in FIGS. 4A and 4B are clear, whereas the 12° C./sec melts shown in FIGS. 3A and 3B are not usable.

A nucleic acid amplification reaction that can produce melts like those shown at 43 and 47 of FIGS. 4A and 4B suitably may be included in a panel for more than one organism and, as such, the variable ramp rate melt information (i.e., fast ramp rates at temperature ranges where no melt signature is expected and a slower ramp rate at a temperature range where a melt signature is expected) suitably may be determined dynamically based on the organism assay(s) in the nucleic acid amplification panel that show positive amplification.

Melt ramps 40 and 44 and derivative curves 41 and 45 of FIGS. 4A and 4B are the same as those shown in FIGS. 3A and 3B at 30 and 34 and 31 and 35. These derivative melt curves were produced from melt data collected at a ramp rate of 2° C./sec and have clear and unambiguous peaks. Likewise, derivative melt curves 43 and 47 also have clear and unambiguous peaks. However, melt ramps 42 and 46 used to produce these derivative melt curves include fast ramps like those of melt ramps 32 and 36 for the approach and finish portions (the temperature was ramped at 6-12° C./sec for the approach and at 12° C./sec for the finish), which suitably may save considerable time in a melt procedure. In order to preserve melt resolution, melt ramps 42 and 46 include a slow ramp portion (approx. 2° C./sec) around the melt transition temperature specific to the amplicon being detected. Melt ramps 42 and 46 include much of the speed of melt ramps 32 and 36, but unlike melt ramps 32 and 36, melt ramps 42 and 46 preserves the high resolution of slow melts with their fast, slow, fast ramping procedure. That is, the dynamic melts have similar melt resolution to industry standard steady ramp rate melts with a consistent ramp rate throughout the melt procedure, while potentially saving considerable time over such industry standard melts.

The total time savings associated with the novel dynamic melt protocol described herein suitably may depend on factors such as, but not limited to, the type of protocol used (e.g., performing a fixed number of cycles prior to a melt vs. performing a melt on one or more analytes as soon as amplification above a selected threshold is detected) and how many analytes are positive. If no analytes are detected (i.e., if the assay results are negative for all analytes), it suitably may be possible to report the negative run results without performing any melt step. As such, the time for melt detection and melt data processing may be deducted entirely from the panel run time. In the case of a FilmArray panel, a negative run with no melt may result in a melt time savings of ~40 seconds. If one or more analytes are detected (i.e., if amplification is detected in one or more sample wells associated with one or more assays), then one or more melts will typically be performed. For a single and three analyte positive assays, for example, total time savings for a FilmArray run is typically ~30 or ~10 seconds, respectively. While these may not be seen as huge time savings, as reaction times become shorter overall and as testing moves into more near-patient settings, any time saved that reduces the time to results is significant. Time savings associated with the novel dynamic melt procedure disclosed herein are discussed in greater detail in Example 6 herein below.

In addition to the foregoing, the novel dynamic melting procedure described herein suitably may allow positive results to be reported out for some assays prior to the full completion of thermocycling, with confirmatory results provided at the end of the test. That is, a dynamic confirmatory melt can be performed as soon as amplification is detected and the results can be reported out. Again, this reduces the time that patients have to wait for results and reduces the amount of time that patients have to spend in the doctor's office. In a singleplex assay (e.g., a COVID-19 assay), a dynamic confirmatory melt can be performed as soon as amplification is detected, the assay run can be stopped, and the results can be reported immediately.

In addition, the novel dynamic melt procedure described herein may have other advantages. For example, melt parameters may be changed for certain assays. For example, if there is a positive detection for an amplicon and the melt includes a relevant mutation (e.g., a mutation associated with anti-microbial susceptibility), the temperature ramping rate in the region of interest (i.e., the region where the melt is expected) may be slowed (e.g., 0.01-2° C./sec, 0.1-1° C./sec, or slower) to facilitate collection of melting data that can show melting shifts and the like that can indicate the presence or absence of relevant mutations in the amplicon population. Such “high resolution” melting and techniques for detection of mutations are described in greater detail in U.S. Pat. No. 9,657,347 (see, e.g., Example 19), U.S. Pat. No. 9,290,663, and U.S. Pat. Pub. No. 2018/0066137, the entireties of which are incorporated by reference herein. Specific examples showing an adaptation of the fast-slow-fast dynamic melt procedure for detection of genetic variations are shown herein below in Example 10. If, however, the positive detection is associated with an amplicon that is not associated with one or more relevant mutations or genetic variations, then the ramping rate in the region of interest suitably may be a more typical value like 2° C./sec-3° C./sec. In another example, the novel dynamic melt procedure described herein suitably may be used to delay melt detection in quantitative or semi-quantitative assays until after one or more internal quantitation standards are detected in order to ensure that the full quantitative range of the assay is accounted for. In yet another example, the novel dynamic melt procedure described herein suitably may be used to shorten the run time for assays with fixed melts after selected numbers of cycles. For example, if a true positive for an organism is expected to be detected, if at all, by melt within the first 15 cycles of PCR, the melt can be omitted if wells for detection of that target organism show positive amplification after the first 15 cycles of PCR.

### Example 2

In this Example, a dynamic melting procedure for one target analyte after a fixed number of amplification cycles is illustrated. In this example, Mycoplasma pneumoniae is the bacterial target and a unique genetic sequence from M. pneumoniae is the amplified amplicon and the target analyte. It is noted that the M. pneumoniae assay is run in triplicate (e.g., in high density array 581), and two of the three wells need to show a positive result for the system to call that organism positive. However, it may also be possible to run some assays in duplicate or singly in some systems. It is also noted that M. pneumoniae is merely an illustrative target and this procedure suitably may be applied to any amplicon in a nucleic acid amplification test-either in a singleplex assay or in a multiplex test or panel.

In a situation where the illustrative analyte (e.g., M. pneumoniae) is the lone analyte, the melt at the conclusion of amplification will be performed based on the melt information for the lone analyte if the lone analyte shows evidence of amplification (e.g., an increase in fluorescence above a threshold value) during the amplification phase. If the lone analyte does not amplify, then the melt suitably may be omitted in order to save time and the assay can be reported as negative without a confirmatory melt. In a situation where the illustrative analyte is one of a plurality of analytes in the test, the post-amplification melt suitably may be based on the analyte(s) that shows evidence of amplification during the amplification phase. In the illustrated Example, that positive analyte is M. pneumoniae, but it could be any one or more analytes included in the assay. If no analyte in the assay shows positive amplification, then the post-amplification melt may be omitted in order to save time-to-result.

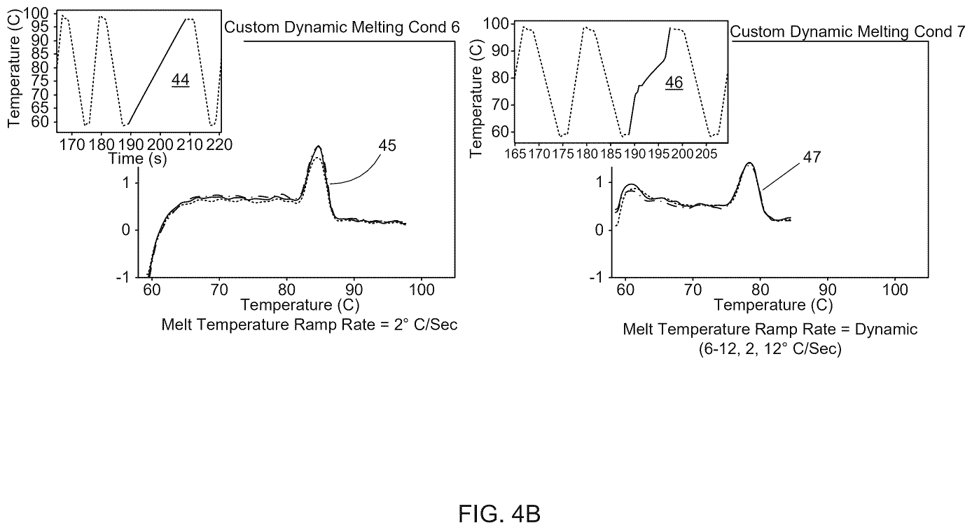

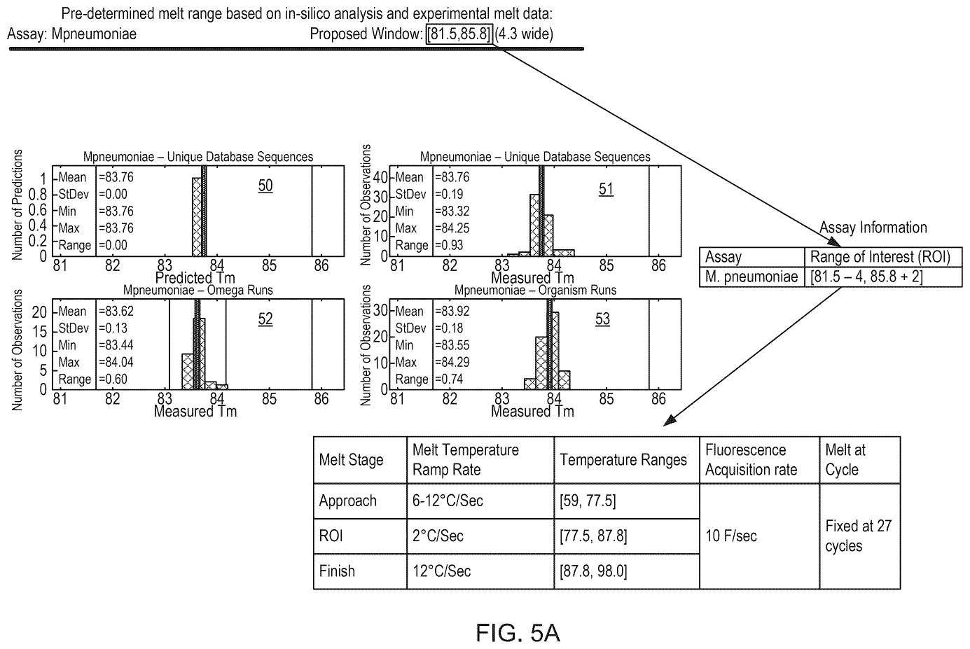

Referring now to FIG. 5A, a procedure for determining the melt characteristics of a target analyte is illustrated. In this Example, the target analyte is an amplicon of M. pneumoniae, but the procedure illustrated in FIG. 5A can suitably be applied to any analyte. FIG. 5A illustrates the determination of the melt range for the M. pneumoniae amplicon based on both ‘in-silico’ analysis (panel 50) and experimentally collected melt data (panels 51-53). Based on these data, it was determined that the M. pneumoniae amplicon used in this test should melt between 81.5° C. and 85.8° C. (Table 1). Also based on these data, the range of interest (ROI) for dynamic melting was determined to be 81.5° C.-4° C. and 85.8° C.+2° C. (Table 1). That is, the M. pneumoniae amplicon used in this test is expected to melt between 81.5° C. and 85.8° C., but a margin suitably may be added to the expected melt range to ensure that the beginning and the end of the melt are included in the slow temperature ramp period and to allow for instrument-dependent factors such as, but not limited to, lag between the fast ramped approach and the slow ramped ROI, variations in the instrument's temperature monitoring system, differences between the temperature of the heater and the fluid temperature of the reaction, and the like.

TABLE 1

| Assay | Range of Interest (ROI) |
|---|---|
| M. pneumoniae | [81.5 − 4, 85.8 + 2] |

As contained in FIG. 5A and as shown in Table 2 below, the approach, which suitably may be ramped at 6-12° C./Sec, ranges from 59° C. to 77.5° C., the ROI, which suitably may be ramped at 2° C./Sec, ranges from 77.5° C. to 87.8° C., and the Finish, which may be ramped at 12° C./Sec, ranges from 87.8° C. up to 98° C. In the cases of each of the Approach, ROI, and Finish, the fluorescence acquisition rate is 10 acquisitions per second (i.e., 10 F/sec) and the melt is performed after a fixed number of cycles PCR at cycle 27, which may be an interim point or the completion of amplification. These numbers are based specifically on M. pneumoniae, but persons of ordinary skill will understand that this procedure and principal can be applied to any amplicon.

TABLE 2

| | Melt | | | Fluorescence | |
|---|---|---|---|---|---|
| Melt | Temperature | | Temperature | Acquisition | Melt at |
| Stage | Ramp rate | | Ranges | rate | Cycle |
| Approach | 6-12° | C./Sec | [59, 77.5] | 10 F/sec | Fixed at 27 |
| ROI | 2° | C./Sec | [77.5, 87.8] | | cycles |
| Finish | 12° | C./Sec | [87.8, 98] | | |

FIG. 5B illustrates the resulting dynamic melt based on these parameters. The left panel of FIG. 5B graphically shows the temperature ramp based on Table 2 at 54. Note the difference between temperature ramp 54 and the steady temperature ramp 30 of FIG. 3A, which are based on melting of the same M. pneumoniae amplicon. The right panel of FIG. 5B shows the derivative melt curve at 55, which is sharp and clear. Compare the derivative melt curve at 55 to the derivative melt curve at 31 of FIG. 3A. As can be seen, dynamic melting ramps and steady melt ramps produce similar high-quality melt curves, while dynamic melting does so with significant time savings.

### Example 3

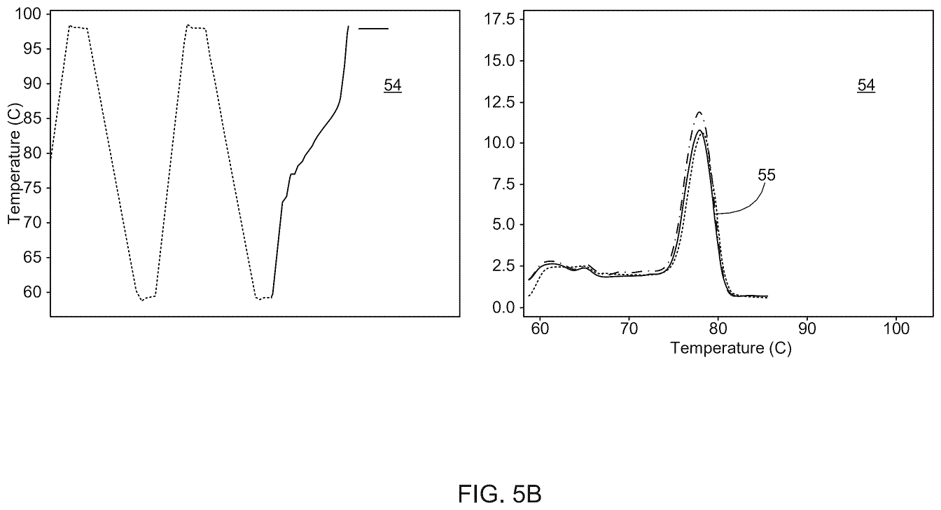

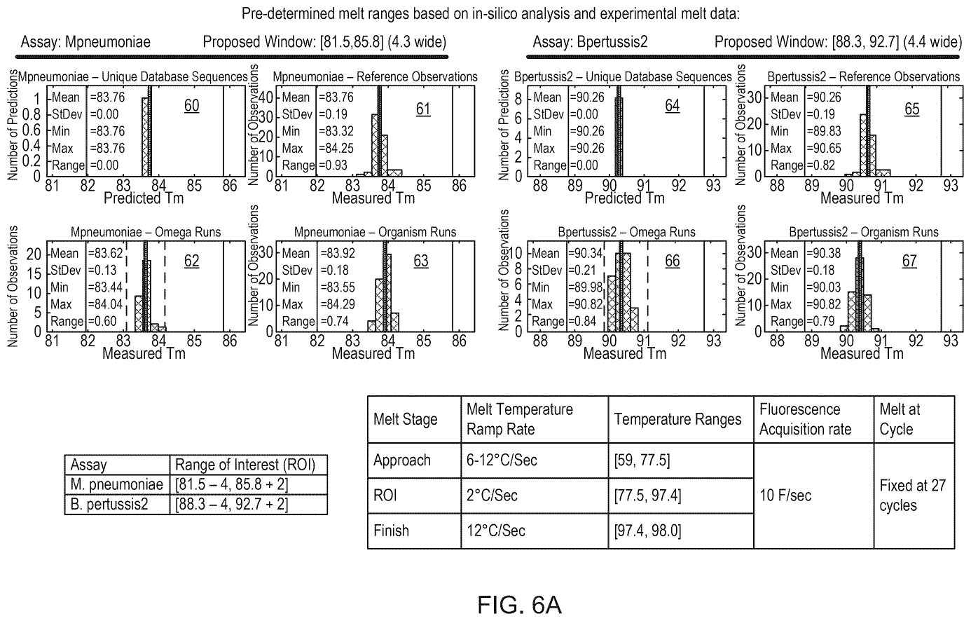

FIGS. 6A and 6B illustrate another example of a dynamic melt procedure with two positive amplicons. The dynamic melt procedure illustrated herein suitably may be applicable to a test with two analytes, or the two analytes illustrated in this Example may be part of a larger test with more than two analytes. If this were a test with multiple analytes, this Example would illustrate a situation where two amplicons showed evidence of amplification and both amplicons need melt confirmation.

In this Example, the analyte amplicons are the M. pneumoniae amplicon discussed in detail in Example 2 and an amplicon for identification Bordetella pertussis (B. pertussis 2). It is noted that the M. pneumoniae and B. pertussis 2 assays are run in triplicate in this Example, and, typically, two of the three wells for each analyte need to show a positive result for the system to call that organism positive. However, it may also be possible to run some assays in duplicate or singly in some systems. In-silico and experimental melt data procedure for determining the melt range of M. pneumoniae and B. pertussis 2 is illustrated in FIG. 6A. The in-silico and experimental melt data used to determine the melt temperature range for M. pneumoniae (panels 60-63) is the same as was illustrated in FIG. 5A and Example 2. Based on these data, it was determined that the M. pneumoniae amplicon used in this test should melt between 81.5° C. and 85.8° C. For B. pertussis 2, panel 64 illustrates an in-silico analysis of the melting range of the B. pertussis 2 amplicon (melting around 90.3° C.) and panels 65-67 illustrate experimental melt data (melting in a range between about 89° C. and 91° C.). Based on these data, it was determined that the B. pertussis 2 amplicon used in this test should melt between 88.3° C. and 92.7° C. (Table 3).

The melt range of interest (ROI) for M. pneumoniae is 77.5° C. to 87.8° C. and the ROI for B. pertussis 2 84.3° C. to 94.7° C. Because these ranges overlap, it was decided in this Example to combine the melt for the two amplicons into one melt. If two or more targets have close or overlapping ROIs and the two or more targets have sufficiently amplified at the same time, it may be faster to observe both melts in a single, combined ROI rather than doing separate melts for each target. Thus, the range of interest (ROI) for dynamic melting these two amplicons in a single melt is 77.5° C. to 94.7° C. That is, the overlapping melt range for the two amplicons includes the low range for M. pneumoniae (i.e., 81.5° C.-4° C.) and the upper range for B. pertussis 2 (i.e., 92.7° C.+2° C.). One will appreciate, however, that this is merely illustrative and that the melt data for M. pneumoniae and B. pertussis 2 can be collected in separate melt steps. Example 5, herein below, illustrates such an experiment.

TABLE 3

| Assay | Range of Interest (ROI) |
|---|---|
| M. pneumoniae | [81.5 − 4, 85.8 + 2] |
| B. pertussis 2 | [88.3 − 4, 92.7 + 2] |

As contained in FIG. 6A and in Table 4 below, the approach, which suitably may be ramped at 6-12° C./Sec, ranges from 59° C. to 77.5° C., the ROI, which suitably may be ramped at 2° C./Sec, ranges from 77.5° C. to 94.7° C., and the Finish, which may be ramped at 12° C./Sec, ranges from 94.7° C. up to 98° C. In the cases of each of the Approach, ROI, and Finish, the fluorescence acquisition rate is 10 acquisitions per second (i.e., 10 F/sec) and the melt is fixed at cycle 27, which suitably may be at the completion of amplification.

TABLE 4

| | Melt | | | Fluorescence | |
|---|---|---|---|---|---|
| Melt | Temperature | | Temperature | Acquisition | Melt at |
| Stage | Ramp rate | | Ranges | rate | Cycle |
| Approach | 6-12° | C./Sec | [59, 77.5] | 10 F/sec | Fixed at 27 |
| ROI | 2° | C./Sec | [77.5, 94.7] | | cycles |
| Finish | 12° | C./Sec | [94.7, 98.0] | | |

These numbers are based specifically on M. pneumoniae and B. pertussis 2, but persons of ordinary skill will understand that this procedure and principal can be applied to any amplicons with overlapping or nearby melt ranges.

FIG. 6B illustrates the resulting dynamic melt based on these parameters. The left panel of FIG. 6B graphically shows the temperature ramp 68 based on the information shown in Table 4. The right panel of FIG. 6B shows the derivative melt curves for M. pneumoniae (69) and B. pertussis 2 (70), which are sharp and clear and similar to the steady state melt curves shown elsewhere herein (see, e.g., FIG. 11B).

### Example 4

Examples 2 and 3 showed dynamic melts taken after a fixed number of cycles of amplification. In this Example, a procedure is shown wherein melt data for an amplicon suitably may be collected as soon as the amplicon shows amplification above a selected threshold value (e.g., a crossing point (Cp) threshold, above a selected relative fluorescence unit value, etc.). This has the potential to save additional time over the cases shown in Examples 2 and 3 because results suitably may be reported as soon as amplification above the given threshold value is detected and the melt is performed.

In this Example, M. pneumoniae is the amplicon—this is the same amplicon that was used exclusively in Example 2 and in combination with B. pertussis 2 in Example 3. It is noted that the M. pneumoniae assay is run in triplicate in this Example, and, typically, two of the three wells for the analyte need to show a positive result for the system to call that organism positive. However, it may also be possible to run some assays in duplicate or singly in some systems. While M. pneumoniae or another single amplicon may be the only analyte in an assay (i.e., this Example suitably may illustrate a singleplex assay), persons of ordinary skill will appreciate that this Example may illustrate a test or panel of assays with many potential analytes (i.e., a multiplex test) where, for example, the M. pneumoniae amplicon is either the only amplicon to amplify or M. pneumoniae is the amplicon for which amplification is first detected. In any case, the procedure described in this Example is equally applicable to a singleplex assay or a multiplex panel or test.

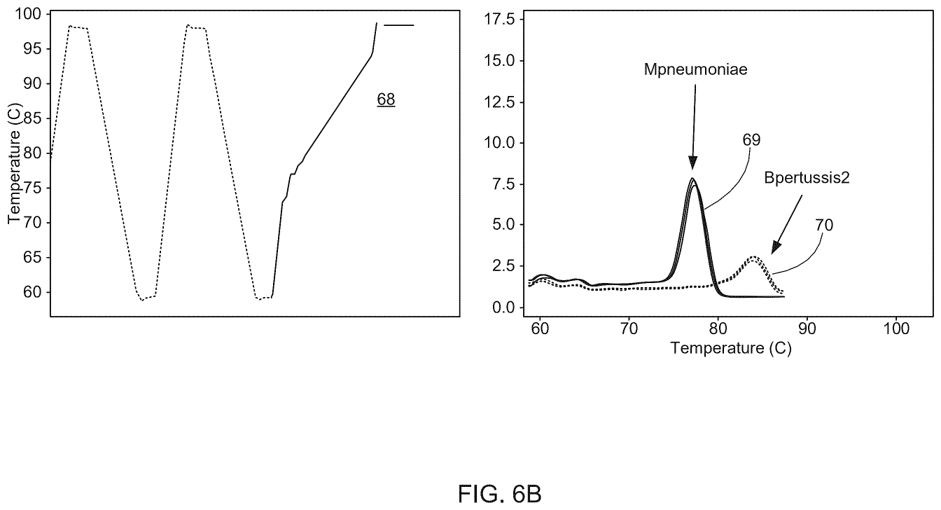

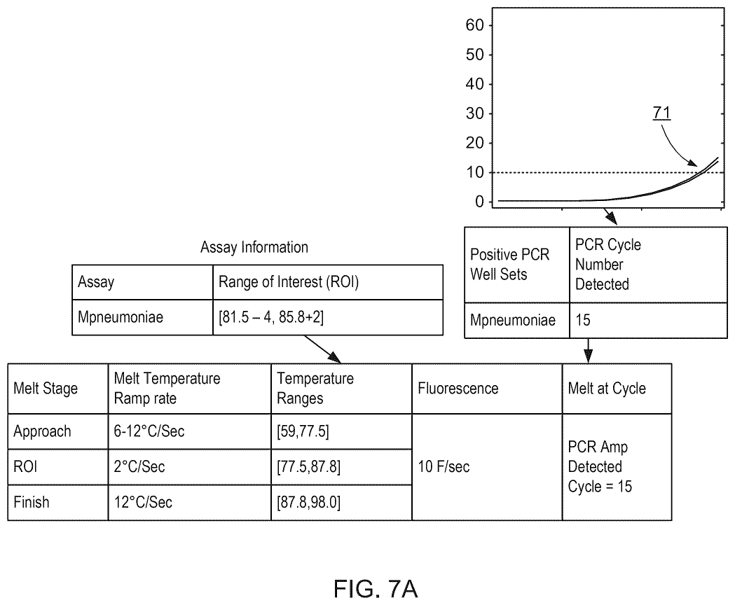

The information shown in Table 1 above is shown again in FIG. 7A. The M. pneumoniae amplicon is expected to melt between 81.5° C. and 85.8° C. and the range of interest (ROI) for dynamic melting is 77.5° C. to 87.8° C. (Table 1). The parameters for the dynamic melt are shown below in Table 5. The information in Table 5 is nearly identical to the information shown in Table 2 herein above.

TABLE 5

| | Melt | | | Fluorescence | |
|---|---|---|---|---|---|
| Melt | Temperature | | Temperature | Acquisition | Melt at |
| Stage | Ramp rate | | Ranges | rate | Cycle |
| Approach | 6-12° | C./Sec | [59, 77.5] | 10 F/sec | PCR Amp |
| ROI | 2° | C./Sec | [77.5, 87.8] | | Detected at |
| Finish | 12° | C./Sec | [87.8, 98.0] | | Cycle 15 |

The parameters for the dynamic melt are: Approach, 59° C. to 77.5° C. (ramped at 6-12° C./Sec), ROI, 77.5° C. to 87.8° C. (ramped at 2° C./Sec), and Finish, 87.8° C. up to 98° C. (ramped at 6-12° C./Sec) (Table 5). The difference between Example 2/Table 2 and the melt in this Example is that the amplicon suitably may be melted after detecting amplification above a selected threshold value instead of waiting until after a fixed number of amplification cycles. In this Example, amplification, as detected by fluorescence, had exceeded a threshold value of 10 RFU (see 71 in FIG. 7A) by amplification cycle 15 and the melt detection was performed at that point. While 10 RFU was used as the threshold value in this case, one will appreciate that other threshold values can be used (see, e.g., FIGS. 3B and 4B, wherein 4 RFU fluorescence data were used). For example, lesser or greater RFU values, crossing point (Cp), Cp plus a selected number of cycles (e.g., 2 additional amplification cycles after Cp is detected), and the like may be used as the threshold value.

The ‘fast-slow-fast’ dynamic temperature ramping profile for melting M. pneumoniae is shown graphically at 72 of FIG. 7B. The melt derivative curves shown at 73 of FIG. 7B are sharp and clear. As in the previous examples, the amplification was performed in triplicate.

In one aspect of the present disclosure, the assay results may be reported positive for the detected organism (in this case, M. pneumoniae) immediately after the confirmatory melt illustrated in FIG. 7B. If the operator is confident that M. pneumoniae is the only positive analyte (e.g., this is a singleplex assay or double positives are statistically unlikely in a panel-based test), the amplification reaction suitably may be stopped after the melt step. In cases where this is possible, this may yield considerable time savings. In another aspect of the present disclosure, the assay results may be reported positive for the detected organism immediately after the melt step and amplification suitably may be continued to the conclusion of amplification (e.g., a fixed number of cycles or until internal quality control standards show positive amplification) to confirm that there are no additional positive detections. If there were additional assay positives in this Example, then one or more additional melts suitably may be performed as the amplification of the assay positives exceeds the given threshold value.

### Example 5

Following on from the previous Example, this Example illustrates a dynamic melt procedure for a test that includes more than one positive amplification and more than one melt. In this Example, the temperature parameters for the dynamic melts suitably may be set dynamically by the assay(s) that is/are positive for a given melt and the cycle(s) at which a melt is performed are also set dynamically according to at which amplification cycle the assay(s) exceed the threshold concentration for detection and melting. In one aspect, if an assay has previously been detected as positive and had a melt performed based on its assay conditions, the temperature range of interest for that assay suitably may not be considered for subsequent melt parameters.

While two assay positives and two dynamic melts are included in this Example, persons of ordinary skill will appreciate that the principal illustrated in this example may be applicable to panel or tests with more than two positives and more than two melts. Likewise, even though the two positives are treated in separate melts due to the fact that the positives exceed the threshold value at different cycles, a suitable scenario suitably may include two or more amplicons in the same melt if, for example, the amplicons exceed the threshold value at the same or nearly the same amplification cycle and if the degree of temperature overlap between the two or more amplicons means that time can be saved by performing one dynamic melt instead of two or more melts.

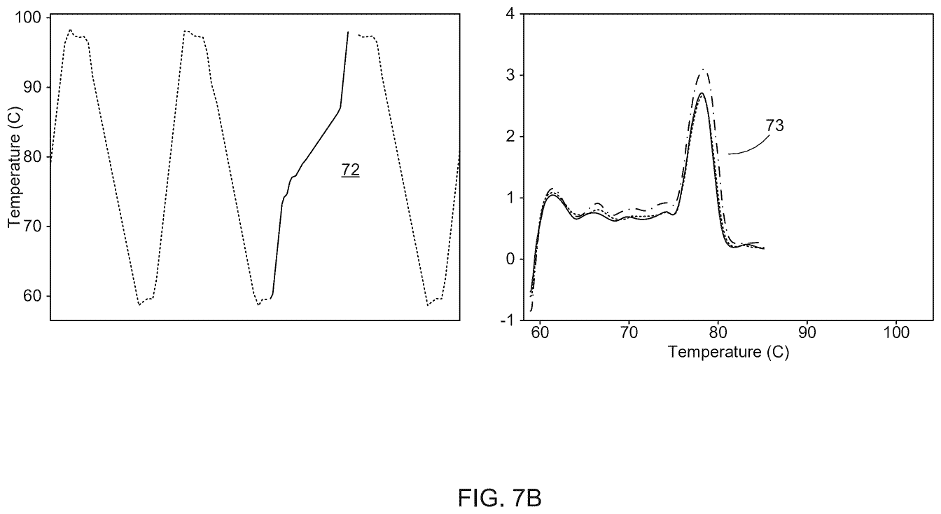

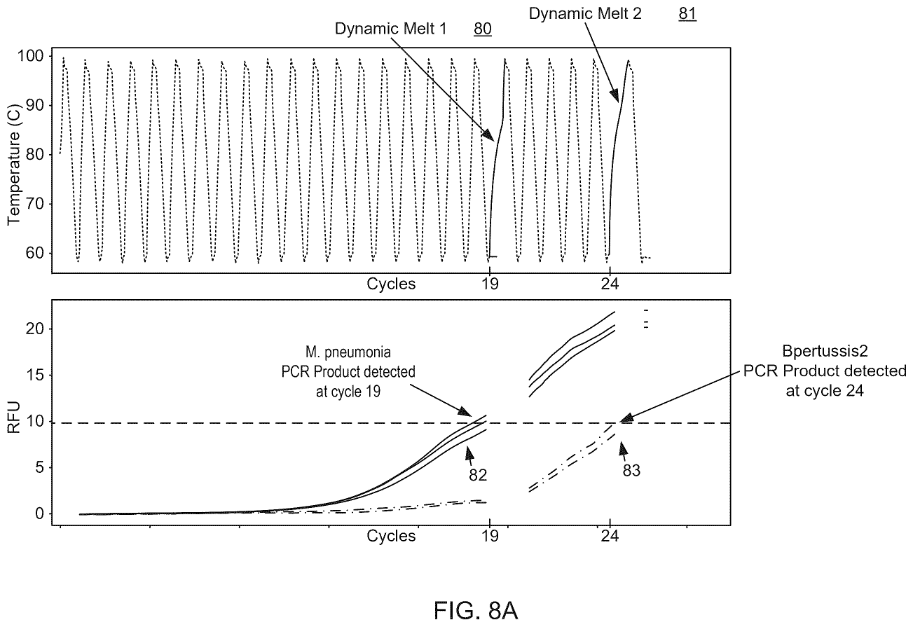

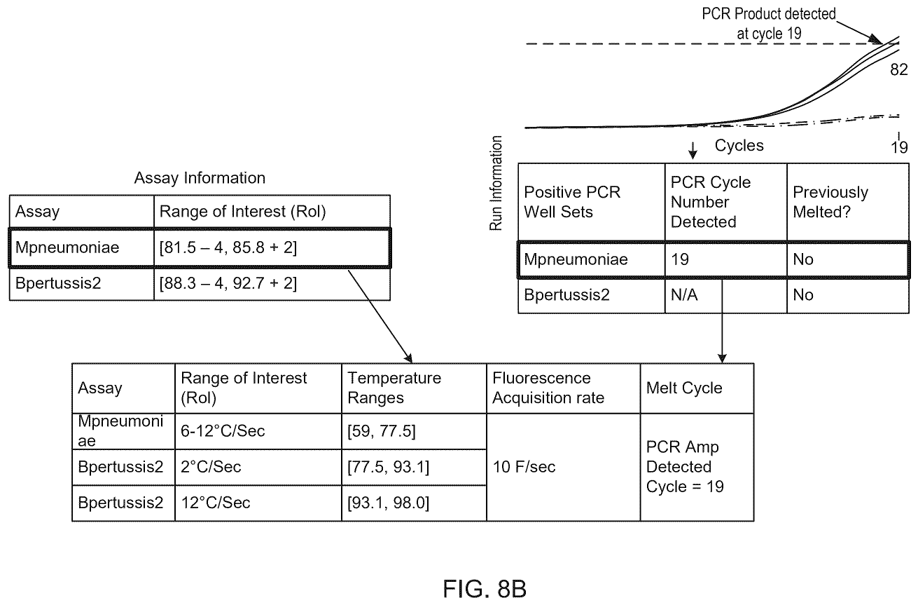

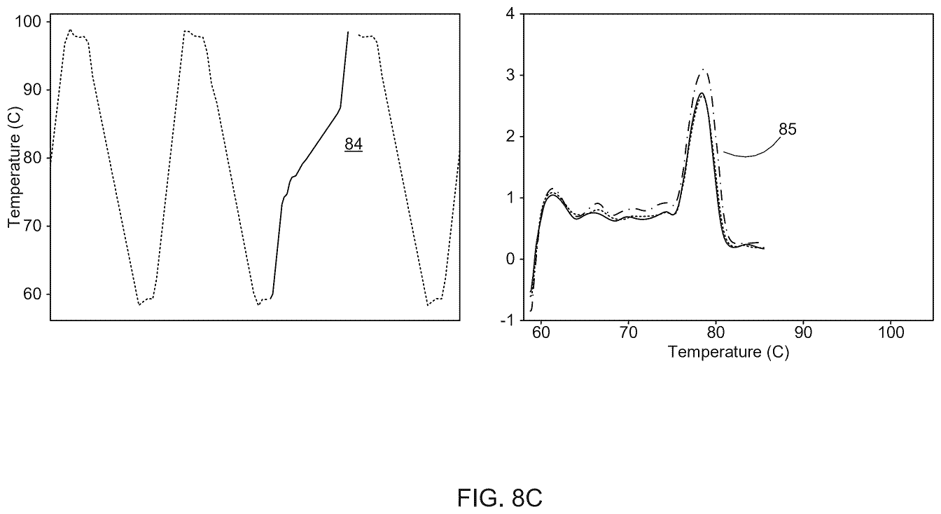

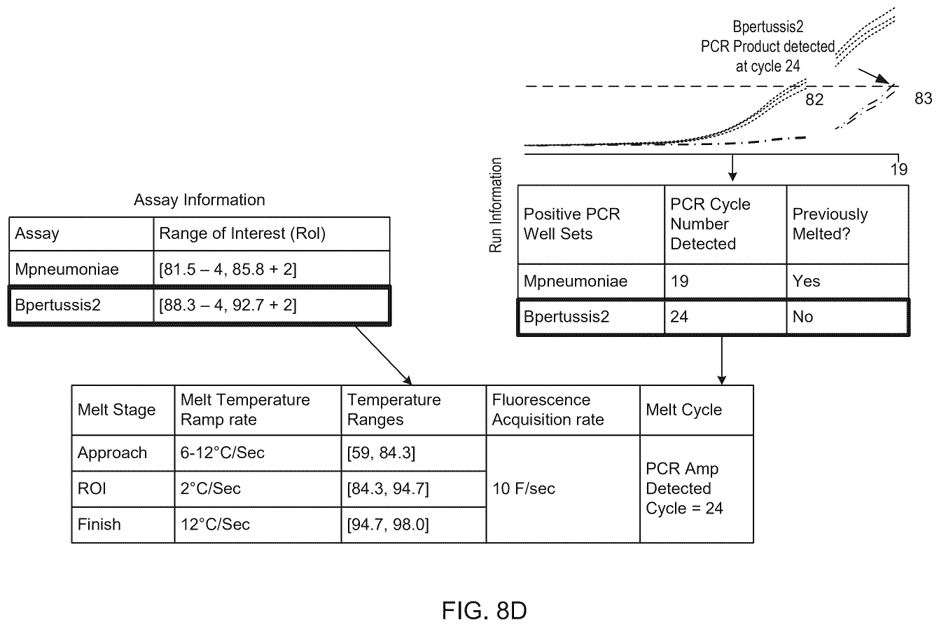

Referring now to FIG. 8A, a thermocycling temperature trace is shown in the upper panel with a first dynamic melt at 80 and, after five additional cycles of PCR, a second dynamic melt at 81. In the lower panel, the real-time fluorescence monitoring of the reaction is illustrated. In the real-time fluorescence trace, it can be seen that the M. pneumoniae amplicon exceeded the threshold (10 RFU, in this case) at cycle 19 (indicated at 82) and the B. pertussis 2 amplicon exceeded the threshold at cycle 24, as indicated at 83. While 10 RFU was used as the threshold value in this Example, one will appreciate that other threshold values can be used (see, e.g., FIGS. 3B and 4B, wherein 4 RFU fluorescence data were used). For example, lesser or greater RFU values, crossing point (Cp), Cp plus a selected number of cycles (e.g., 2 additional amplification cycles after Cp is detected), and the like may be used as the threshold value. As in the previous Examples, these analytes were run in triplicate and each of the three wells for each amplicon reached the threshold value at approximately the same time, as would be expected if the reagent concentrations (e.g., primer, polymerase, etc.) and the template loading concentration were approximately the same for each assay well.

Referring to FIGS. 8B and 8C, the dynamic melt procedure for the M. pneumoniae amplicon is illustrated. This is largely the same as the M. pneumoniae amplicon melting procedure that was illustrated in Example 5. In Example 5, the “PCR informed” melt of the M. pneumoniae amplicon was performed at cycle 15. In this Example the M. pneumoniae melt was performed at cycle 19. Such test-to-test variability is not unexpected and is adapted for in a novel way in the present disclosure. The M. pneumoniae amplicon is expected to melt between 81.5° C. and 85.8° C. and the range of interest (ROI) for dynamic melting is 77.5° C. to 87.8° C. (see, e.g., Table 1). The parameters for the dynamic melt are: Approach, 59° C. to 77.5° C. (ramped at 6-12° C./Sec), ROI, 77.5° C. to 87.8° C. (ramped at 2° C./Sec), and Finish, 87.8° C. up to 98° C. (ramped at 12° C./Sec). The ‘fast-slow-fast’ dynamic temperature ramping profile for melting the M. pneumoniae amplicon is shown graphically at 84 of FIG. 8C. The resultant melt derivative curves for the triplicate reaction are shown at 85 of FIG. 8C. As in the previous examples, the derivative melt curves are sharp and clear.

Referring now to FIGS. 8D and 8E, the dynamic melt procedure for the B. pertussis 2 amplicon is illustrated. The melt temperatures, ROI, and dynamic melt procedure for this B. pertussis 2 amplicon are nearly the same as was illustrated in reference to FIGS. 6A and 6B, except the timing of the melt cycle was ‘PCR-informed’ and based on the cycle when the B. pertussis 2 amplicon concentration exceeded the threshold value at cycle 24. In addition, in Example 3/FIGS. 6A and 6B, the M. pneumoniae and B. pertussis 2 amplicons were melted in one melt, whereas they are melted separately in this Example.

The melt range for the B. pertussis 2 amplicon 88.3° C. to 92.7° C. and the ROI for B. pertussis 2 amplicon is 84.3° C. to 94.7° C. The parameters for the dynamic melt are: Approach, 59° C. to 84.3° C. (ramped at 6-12° C./Sec), ROI, 84.3° C. to 94.7° C. (ramped at 2° C./Sec), and Finish, 94.7° C. up to 98° C. (ramped at 12° C./Sec). The ‘fast-slow-fast’ dynamic temperature ramping profile for melting the B. pertussis 2 amplicon is shown graphically at 86 of FIG. 8E. The resultant melt derivative curves for the triplicate reaction are shown at 87 of FIG. 8E. As in the previous examples, the derivative melt curves for the triplicate reaction are sharp and clear.

### Example 6

As illustrated in the Examples presented herein, dynamic melting can give the same melt quality as slow, steady state melting while also offering time savings over such steady state melting. Fast, steady state melting (e.g., collecting melt data while ramping at 12° C./sec) is not feasible. This is demonstrated on high, medium, and low signal melt curves.

The type of protocol used (fixed cycle, or amplification detection informed) and how many analytes are positive, determines the time savings. For a negative run (i.e., no assay positives are detected during amplification), the melt step suitably may be omitted entirely. In a FilmArray run, for example, the melting procedure takes about 40 seconds. Therefore, omitting the melt step in the case of a negative run reduces the time-to-result by about 40 seconds. The time savings for fixed cycle dynamic melting (see, e.g., Examples 2 and 3) is about 30 seconds versus the time needed for a typical FilmArray run. The time savings for detection informed dynamic melting (see, e.g., Examples 4 and 5) is about 10 seconds versus the time needed for a typical FilmArray run. Of course, the time savings for detection informed dynamic melting can be greater if results can be reported as soon as an assay positive is detected and the melt is performed. If results are reported mid-run and if no additional assay positives are detected during the run, no end-of-run melt is needed and the interim report can be treated as the final report.

As PCR gets faster and the time spent in melting becomes an increasingly larger portion of the run time, the dynamic melting procedures described herein and the time savings that they yield become more significant. The dynamic melt procedures described herein save significant time while preserving high quality melt resolution by suitably configuring the instrument to ramp the temperature rapidly in areas (e.g., the approach area and finish area) of the melt temperature ramp where no melt information is expected and to ramp more slowly in a region of interest (ROI) where the melt transition of the relevant amplicon is expected.

### Example 7

The FilmArray Blood Culture Identification (BCID) system is designed to provide rapid identification of a broad range of microorganism pathogens directly from blood culture. The illustrative BCID panel detects the most common bacteria and yeast isolated from positive aerobic blood cultures (PABC), as well as select antibiotic resistance genes, with ≥ 95% sensitivity. A commercial BCID panel is available from BioFire Diagnostics, Inc. The BCID panel and associated methods are described in detail in U.S. Pat. No. 10,053,726, the entirety of which is incorporated herein by reference. This Example describes an adaptation of the methods described in U.S. Pat. No. 10,053,726 using the dynamic melt procedures described herein for distinguishing between true positives and environmental contamination.

Within the FilmArray instrument, subsequent to sample prep, the first-stage multiplex PCR mixture suitably may be amplified in blister 564 for a selected number of amplification cycles (e.g., 20-30 cycles). After first-stage PCR is complete, the mixture suitably may be diluted and transferred to each of the second-stage wells 582. The second-stage PCR reactions suitably may be subjected to amplification conditions (e.g., 63° C. for 19 seconds to 94° C. for 0 seconds) for an additional number of cycles. Melts, in this illustrative example, suitably may be performed after fixed numbers of cycles (e.g., after cycles 20, 26, and 32 cycles) or melts may be ‘PCR-informed’ and suitably may be performed when wells show evidence of amplification exceeding a threshold value. In either case, melts suitably may be performed using the dynamic melt procedure described herein where temperature ramping suitably may be defined by the identity of the organism amplicons in the well(s) that show positive amplification. An amplification assay suitably may be called positive if a melt curve for a given well shows a melt peak (negative first derivative of the melt curve) in a pre-defined temperature range for each second-stage assay.

In the BCID panel, each organism tested in the panel may be ‘binned’ according to expected concentration in the sample and contamination risk, in order to minimize both false positives and false negatives. The organisms were assigned as follows in Table 6:

TABLE 6

| Bin 1 | Bin 2 | | Bin 3 |
|---|---|---|---|
| A. baumannii | Enterococcus | K. pneumoniae | C. albicans |
| E. coli | L. monocytogenes | K. oxytoca | C. glabrata |
| E. cloacae | Staphylococcus | S. marcescens | C. krusei |
| Enterobacteriaceae | S. aureus | P. aeruginosa | C. parapsilosis |
| Proteus | Streptococcus | N. meningitidis | C. tropicalis |
| | S. agalactiae | mecA | |
| | S. pneumoniae | KPC | |
| | S. pyogenes | vanA/B | |
| | H. influenzae | | |

In general, Bin 1 suitably may contain targets that are present at the highest titers in the sample and that are also present as background organisms and are at the highest risk for unexpected positives. Bin 2 suitably may contain target organisms present at high titers in the sample and that have a low presence as background organisms and are a medium risk for unexpected positives. Bin 3 suitably may contain targets present at low titers in positive aerobic blood cultures and that have low to no presence as background organisms and have a low risk for unexpected positives. While the organisms binned in this Example are specific to the BCID panel and to positive aerobic blood cultures, persons of ordinary skill in the art will appreciate that the principle illustrated herein can be applied to any sample with an expected broad range of organism concentrations and/or risk of false positives and false negatives due to environmental and commensal organisms in the sample. Other examples where this approach may be helpful include, but are not limited to, stool samples for diagnosis of GI infections and cerebrospinal fluid for diagnosis of central nervous system infections.

In general, organisms in Bin 1, if they are present in the sample, may be expected to amplify early (e.g., within the first 10-22 cycles of amplification). Thus, if they are present, Bin 1 organisms suitably may be expected to show evidence of amplification above the threshold within, for example, the first 10-22 cycles of amplification. If one or more organisms in Bin 1 amplify in this early phase and if a melt curve for a given Bin 1 well shows a melt peak in a temperature range pre-defined for the organism amplicon, then the assay may be positive. However, because Bin 1 organisms may be at the greatest risk for false positives (due to environmental contamination or other factors), later evidence of amplification (e.g., after 20-22 cycles of amplification) suitably may be considered a false positive and suitably may not trigger a melt detection. That is, if a well for a Bin 1 organism amplifies early (e.g., before amplification cycle 20-22), it suitably may trigger a dynamic melt (either at the time that amplification above a threshold value is detected (e.g., 10 RFU or above) is detected or after a fixed number of amplification cycles (e.g., 15-20 amplification cycles)) and such an amplification may be called positive if the melt curve shows a melt peak in a temperature range pre-defined for the organism amplicon in that well. In contrast, if a well for amplification of a Bin 1 organism amplicon amplifies later (e.g., at cycle 25), such an amplification suitably may not trigger a melt as such amplification may be a false positive and may, for example, be attributed to contamination (e.g., environmental contamination). One will appreciate that the cycle numbers presented in this Example are merely illustrative and that the novel dynamic melting procedure described herein allows a nuanced approach to cycle cut-offs for true positives vs. false positives. Factors such as, but not limited to, the organism titer in a sample and the amount of sample loaded may vary and, as such, cycle cut-offs suitably may also vary. For example, if everything seems to be amplifying early, the cut-off between true and false positives may be moved earlier as well. Likewise, if everything seems to be amplifying late, the cut-off between true and false positives may be moved later.

In general, organisms in Bin 2, if they are present in the sample, may be expected to amplify at a mid-point in amplification (e.g., after about 18-22 cycles but before about 25-27 cycles). One will appreciate, however, that some true positives for Bin 2 organisms may amplify earlier (i.e., before about 18-22 cycles of amplification). If one or more organisms in Bin 2 amplify in this mid-point phase and if a melt curve for a given Bin 2 well shows a melt peak in a temperature range pre-defined for the organism amplicon, then the assay may be positive. However, because Bin 2 organisms still present some risk of false positives due to contamination, later evidence of amplification (e.g., after about 27 cycles of amplification) suitably may be considered a false positive and suitably may not trigger a melt detection. While some true positives for Bin 2 organisms may appear later in the amplification, looking for true positives at an amplification mid-point suitably may capture most true positives while reducing the risk false positives from environmental contamination. The note above about the cycle numbers being merely illustrative and that the dynamic melting procedure suitably may provide a more nuanced approach than strict cut-offs applies here as well.

In general, organisms in Bin 3, if they are present in the sample, may be expected to amplify in the last stage of amplification (e.g., after about 25-27 cycles). One will appreciate, however, that some true positives for Bin 3 organisms may amplify earlier (i.e., before about 25-27 cycles of amplification). If one or more organisms in Bin 3 amplify in this last phase of amplification and if a melt curve for a given Bin 2 assay well shows a melt peak in a temperature range pre-defined for the organism amplicon, then the assay may be positive. Because Bin 3 organisms present a low risk of false positives due to contamination, there is little risk associated with calling a positive even after 32-35 cycles of amplification. The note above about the cycle numbers being merely illustrative and that the dynamic melting procedure suitably may provide a more nuanced approach than strict cut-offs applies here as well.

For an illustrative example, let us examine E. coli, which is a Bin 1 organism (see, Table 6). A true sepsis positive in PABC for E. coli typically grows to a high titer (e.g., >108 cfu/ml) and is expected to be detectable by amplification in the illustrative BCID panel prior to amplification cycle 20 and often before amplification cycle 10. However, E. coli is nearly ubiquitous in the environment and can, for example, often be introduced into a blood sample and ultimately into blood culture bottle via environmental sources and/or media raw materials. For the illustrative BCID panel, it was found that E. coli amplification later than about amplification cycle 18-22 (e.g., prior to cycle 20) was likely due to some type of contamination. Assays for E. coli amplifying prior to about amplification cycle 20 can be treated as true positives and suitably may trigger a melt detection. In contrast, an E. coli positive later than about cycle 20 may be treated as a false positive and suitably may not trigger a melt detection.

For another illustrative example, let us examine an antimicrobial resistance (AMR) gene that may be detected in the illustrative BCID panel. The illustrative BCID and BCID2 panels are capable of detecting a number of different anti-microbial resistance (AMR) markers and genes. In one example, the illustrative BCID panel is capable of detecting the mecA/C gene that is associated with methicillin resistance in S. aureus. In this example, S. aureus is a Bin 2 organism and true positives from PABC may be expected to amplify at a mid-point of amplification (e.g., around 20-25 cycles). If S. aureus is detected without co-detection of the mecA/C gene, then a confirmatory melt suitably may be performed at a ramp rate of ~2° C./sec in the range of interest (ROI), as shown in the Examples presented herein. If, however, amplification is detected in a sample well designated for S. aureus and in a separate well designated for the mecA/Cgene (i.e., S. aureus and mecA/C are co-detected), then a confirmatory melt suitably may be performed for both S. aureus and mecA/C. Depending on their respective melt temperature ranges and melt ramp rates needed for optimal detection and whether or not amplification is detected for the two at the same or nearly the same cycle or if they are temporally separated, S. aureus and mecA/C confirmatory melts may be performed together or separately. If S. aureus is detected and a confirmatory melt is performed and then, at a later cycle, the mecA/Cgene is detected, a confirmatory melt for mecA/C may be performed. If, however, S. aureus amplification is not detected and the mecA/C gene is detected by amplification, a melt for the mecA/C gene suitably may be omitted because the loci must be from another organism and not contributory to methicillin resistant S. aureus. In other words, a melt for an AMR gene or markers suitably may not be performed until the amplification of the associated pathogen is detected. This can potentially save time that might otherwise be spent in melting analysis and may make AMR detection more robust and conclusive.

Additionally, it is understood that the information obtained for one organism can be used to assist with positive or negative calls for other organisms, particularly if there is some cross-reactivity between the organisms, or if there is some other relationship such as a bacterium and an antibiotic resistance gene associated with that bacterium. In Table 6 above it can be seen that Enterococcus (“Entero”) and Staphylococcus (“Staph”) are both Bin 2 organisms and, if present, amplification for each should be detected between about 18-27 cycles of amplification. However, in many known assays for Entero, due to similarities in target sequence, there is cross-reactivity with Staph, thereby potentially causing a late Cp in a true negative Entero sample that is positive for Staph. To reduce the effect of potential cross-reactivity for the Entero assay in such a situation where cross-reactivity is an issue, a positive or negative call for Staph may be made by amplification and melt if Staph amplification is detected between about 18-27 cycles of amplification. If Staph is positive, thereby affecting the Entero sample, Entero could be called based on an earlier result, illustratively if amplification of Entero is detected prior to amplification cycle 20-22. If Staph is negative, then the Entero assay would be unaffected and the call may be made, if at all, if Entero amplification is detected between about 18-27 cycles of amplification, or whichever cycle range was chosen as optimized for that assay without cross-reactivity. It is noted, however, that in blood culture, positive growth is based on the combined organism growth of all organisms that are present, and one or more organisms may be present at amounts lower than either would be from a single infection. The binning and cycle range at which a cross-reactive assay is called may need to be adjusted accordingly. By adjusting the binning and cycle range used for the call of the cross-reactive assay based on a positive or negative call from the other assay, cross-reactivity issues from double infection samples can be called accurately, illustratively without the need to redesign the primers to avoid cross-amplification.

While the organisms described in this example were categorized into three bins and early, middle, and late melt cycle ranges were used in this example for each of the bins, it is understood that any number of bins and melt cycles may be used and that any cycle may be chosen as a melt cycle. Separation between false positives and false negatives may be achieved with only two bins and/or two melt cycles in some assays, whereas four or more bins and/or melt cycles may be needed in other assays. Further, while the example used samples from culture, it is understood that multiple bins and/or melt cycles may be appropriate for assays using uncultured materials.

It is understood that, while the above example identifies organisms, it is understood that the same methods and devices may be used to identify different target sequences in one or several organisms by amplifying different loci of that organism.

### Example 8

Certain embodiments of the present invention may also involve or include a PCR system configured to make positive or negative calls from amplification curves or melt curves or a combination thereof. Illustrative examples are described in U.S. Pat. No. 8,895,295, already incorporated by reference, for use with pouch 510 or similar embodiments. However, it is understood that the embodiments described in U.S. Pat. No. 8,895,295 are illustrative only and other systems may be used according to this disclosure. For example, referring to FIG. 15, a block diagram of an illustrative system 700 that includes control element 702, a thermocycling element 708, and an optical element 710 according to exemplary aspects of the disclosure is shown.

In at least one embodiment, the system may include at least one PCR reaction mixture housed in sample vessel 714. In certain embodiments, the sample vessel 714 may include a PCR reaction mixture configured to permit and/or effect amplification of a template nucleic acid. Certain illustrative embodiments may also include at least one sample block or chamber 716 configured to receive the at least one sample vessel 714. The sample vessel 714 may include any plurality of sample vessels in individual, strip, plate, or other format, and, illustratively, may be provided as or received by a sample block or chamber 716.

One or more embodiments may also include at least one sample temperature controlling device 718 and/or 720 configured to manipulate and/or regulate the temperature of the sample(s). Such a sample temperature controlling device may be configured to raise, lower, and/or maintain the temperature of the sample(s). In one example, sample controlling device 718 is a heating system and sample controlling device 720 is a cooling system. Illustrative sample temperature controlling devices include (but are not limited to) heating and/or cooling blocks, elements, exchangers, coils, radiators, refrigerators, filaments, Peltier devices, forced air blowers, handlers, vents, distributors, compressors, condensers, water baths, ice baths, flames and/or other combustion or combustible forms of heat, hot packs, cold packs, dry ice, dry ice baths, liquid nitrogen, microwave- and/or other wave-emitting devices, means for cooling, means for heating, means for otherwise manipulating the temperature of a sample, and/or any other suitable device configured to raise, lower, and/or maintain the temperature of the sample(s).

The illustrative PCR system 700 also includes an optical system 710 configured to detect an amount of fluorescence emitted by the sample 714 (or a portion or reagent thereof). Such an optical system 710 may include one or more fluorescent channels, as are known in the art, and may simultaneously or individually detect fluorescence from a plurality of samples.

At least one embodiment of the PCR system may further include a CPU 706 programmed or configured to operate, control, execute, or otherwise advance the heating system 718 and cooling system 720 to thermal cycle the PCR reaction mixture, illustratively while optical system 710 collects fluorescent signal. CPU 706 may then generate an amplification curve, a melt curve, or any combination, which may or may not be printed, displayed on a screen, or otherwise outputted. Optionally, a positive, negative, or other call may be outputted based on the amplification and/or melt curve. Optionally only the calls are outputted, illustratively one call for each target tested.

Additional examples of illustrative features, components, elements, and or members of illustrative PCR systems and/or thermal cyclers (thermocyclers) are known in the art and/or described above or in U.S. Pat. Pub. Nos. 2014/0273181 and 2020/0319441 and U.S. Pat. No. 10,698,190, the entireties of which are herein incorporated by reference.

### Example 9

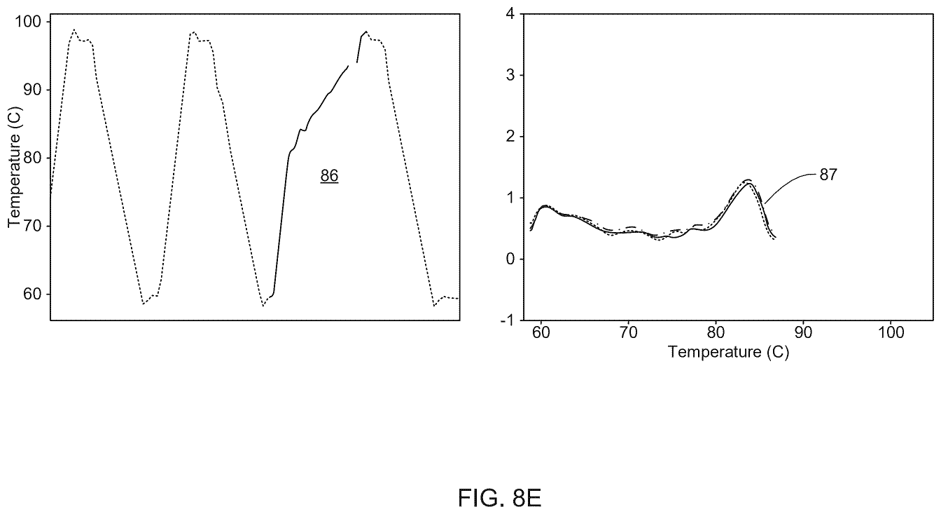

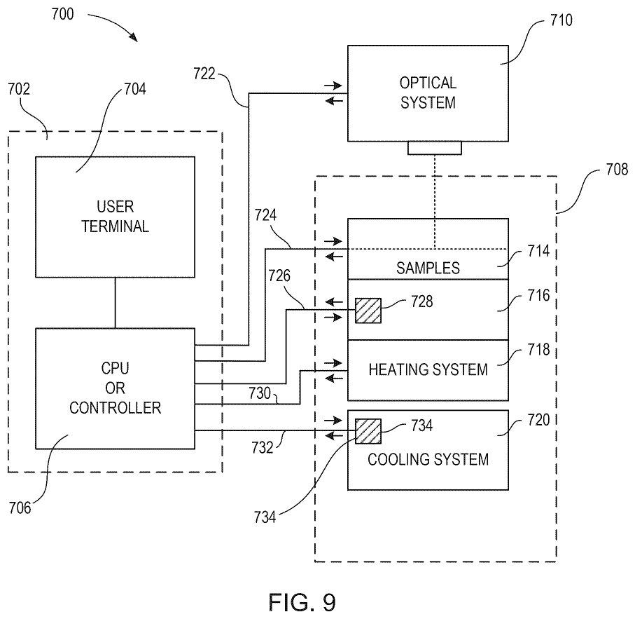

FIG. 10 illustrates a flow diagram of an example dynamic melt detection method 1000 for calling a sample for a target nucleic acid sequence. The method 1000 may be implemented by various components of the PCR system as described below with reference to FIG. 9, including a processor or controller, an optical element, and one or more temperature controlling devices, such as thermocycling elements including a heating system and a cooling system. In some embodiments, the method 1000 or a portion thereof may be implemented in a set of instructions and stored on a computer-readable memory and executable on one or more processors or a controller.

A portion of a sample is included in a sample well along with primers for amplifying the target nucleic acid sequence and a fluorescent dye such as a dsDNA binding dye. Portions of the sample may be included in multiple sample wells where each sample well includes primers for amplifying a different target nucleic acid sequence from a different organism. Each target nucleic acid sequence has a different characteristic melt temperature range. For example, the characteristic melt temperature range for the M. pneumoniae amplicon is 77.5° C. to 87.8° C., while the characteristic melt temperature range for the B. pertussis 2 amplicon is 84.3° C. to 94.7° C.

At block 1002, the sample in each of the sample wells is amplified via thermal cycling which includes at least a two-step PCR protocol. The PCR protocol may include for each of several cycles, an in-cycle temperature adjusting segment or denaturation segment where the sample well is heated from an annealing temperature to a denaturation temperature and cooled from the denaturation temperature to the annealing temperature. The PCR protocol may also include for each of the several cycles, an extension segment where the temperature is held constant at an optimal temperature for DNA polymerase activity. The PCR protocol may also omit a specific elongation temperature hold. As explained in greater detail elsewhere herein, the DNA polymerase may be active and may complete elongation of the primers while the temperature ramps up from the annealing temperature to the denaturation temperature. In some embodiments, the processors or controller provide control signals to a thermocycling element to heat the sample well to the denaturation temperature using an initial ramp rate, to cool the sample well to the annealing temperature, and to hold the temperature of the sample well constant. The initial ramp rate may be a fixed ramp rate (e.g., 12° C./sec for each of the cycles).

Then at block 1004, fluorescent data (which indicates an amount of fluorescence emitted by the sample) is collected from the sample at each of the sample wells during the in-cycle temperature adjusting segment for each of N cycles, where N is 1, 2, 3, 4, 5, 6, or more cycles. The fluorescent data may be collected by an optical system such as the optical system 710 as described above with reference to FIG. 9 and provided to the processors or controller. For example, the optical system may provide light to the sample (e.g., from an LED) at each of the sample wells and may include optical detectors to detect the amount of fluorescent signal produced by the sample at each of the sample wells. In some embodiments, the processors or controller provide control signals to the optical system 710 to detect the amount of light scattered by the sample at each of the sample wells during the in-cycle temperature adjusting segment for each of the N cycles. The processors or controller may then collect an amount of fluorescence along with a temperature of the sample (temperature, fluorescence pairs) at several points in time during each of the N cycles. It is understood that fluorescence is illustrative only, and other ways of measuring and detecting amplification are within the scope of this disclosure.

At block 1006, the processors or controller may determine whether the amount of fluorescence at any of the sample wells exceeds a threshold. The threshold may be an amount greater than or equal to a limit-of-detection value for concluding a rise in the concentration of nucleic acid in the sample well. The threshold may also be a crossing point (Cp), a selected number of relative fluorescence units (RFUs), a Cp plus a predetermined number of additional cycles of amplification, or any suitable threshold.

If the amount of fluorescence at one of the sample wells exceeds the threshold, the processors or controller may identify the target nucleic acid sequence for the sample well and the characteristic melt temperature range for the target nucleic acid. For example, if the sample well includes the M. pneumoniae amplicon, the characteristic melt temperature range may be 77.5° C. to 87.8° C. The processors or controller may be programmed with information about each of the sample wells in the assay including, but not limited to, the identity of the target in each well and the characteristic melt temperature range for each target.

Then the processors or controller determine an adjusted ramp rate profile for heating the sample well based on the characteristic melt temperature range for the target nucleic acid being amplified in the sample well (block 1008). The adjusted ramp rate profile may include different ramp rates for heating the sample well from the annealing temperature to the denaturation temperature. At least one of the ramp rates in the adjusted ramp rate profile may be faster than the initial ramp rate to increase the speed of a melt at temperatures where no melt signature is expected from the reaction. For example, the adjusted ramp rate profile may include a first ramp rate for an approach temperature range, a second ramp rate for a characteristic melt temperature range, and a third ramp rate for a finish temperature range. The approach temperature range may be from the annealing temperature to an initial melt temperature (e.g., 59° C. to 77.5° C.) at the beginning of the characteristic melt temperature range. The characteristic melt temperature range may be from the initial melt temperature to a final melt temperature (e.g., 77.5° C. to 87.8° C.). The characteristic melt temperature range may also be referred to as the melt ROI. The finish temperature range may be from the final melt temperature to the denaturation temperature (e.g., 87.8° C. to 98° C.).

The first ramp rate and the third ramp rate may be faster than the second ramp rate to increase the speed of a melt at temperatures where no melt signature is expected from the reaction. For example, the first ramp rate may be between 6 and 12° C./sec, and the third ramp rate may be 12° C./sec. The second ramp rate may be similar to the initial ramp rate (e.g., 2° C./sec) to slow down the melt at temperatures where a melt signature is expected from the reaction.

Then at block 1010, a melt detection is performed using the adjusted ramp rate profile. For example, during a subsequent thermal cycle, the processors or controller provide control signals to the thermocycling element to heat the sample well to the denaturation temperature using the adjusted ramp rate profile, to cool the sample well to the annealing temperature, and to hold the temperature of the sample well constant. Then a melting curve is generated based on fluorescence data detected during the melt detection. The melting curve is then analyzed to identify the target nucleic acid of the plurality of target nucleic acids corresponding to the sample well. For example, the melt peak may indicate the organism within the sample well. In a specific example, if the sample well includes the M. pneumoniae amplicon, and the melt peak is indicative of M. pneumonia, then the processors or controller may identify that the sample includes M. pneumonia.

In some embodiments, the processors or controller may determine that the amount of fluorescence at multiple sample wells exceeds the threshold, where each of the sample wells includes primers for amplifying a different target nucleic acid sequence from a different organism. For example, the processors or controller may determine that the amount of fluorescence at a first sample well having primers for amplifying a first target nucleic acid having a first characteristic melt temperature range exceeds the threshold, and that the amount of fluorescence at a second sample well having primers for amplifying a second target nucleic acid having a second characteristic melt temperature range also exceeds the threshold.

Then the processors or controller may generate a first adjusted ramp rate profile for the first sample well corresponding to the first characteristic melt temperature range and a second adjusted ramp rate profile for the second sample well corresponding to the second characteristic melt temperature range. In some embodiments, the processors or controller may perform a first melt detection using the first adjusted ramp rate profile at a first melt cycle. Then the processors or controller may perform a second melt detection using the second adjusted ramp rate profile at a second melt cycle which may occur after the first melt cycle. For example, the second melt cycle may occur five cycles after the first melt cycle.

In other embodiments, the processors or controller may combine the first and second adjusted ramp rate profiles into a combined ramp rate profile and may perform a melt detection using the combined ramp rate profile. For example, if the first characteristic melt temperature range is from 77.5° C. to 87.8° C. and the second characteristic melt temperature range is from 84.3° C. to 94.7° C., the processors or controller may generate a combined characteristic melt temperature range of 77.5° C. to 94.7° C. Then the processor or controller may generate the combined ramp rate profile using the melt ROI of 77.5° C. to 94.7° C.

### Example 10

In this Example, dynamic melting procedures for detecting the presence of genetic sequence variants are described. Copies of double-stranded nucleic acid generated during a nucleic acid amplification reaction (called products or amplicon) will have unique sequences based on the template that was amplified. Amplicon length and sequence/composition (typically, A-T/G-C content) determines the temperature at which the double-stranded DNA will melt apart, which is known as the melting temperature (Tm) of the amplicon. Products made from different targets will have different sequences and, therefore, different Tms and, in many cases, different melting curve shapes. As such, a nucleic acid binding dye added to an amplification reaction can be used for nucleic acid melting analysis due to the property of nucleic acid binding dyes wherein they fluoresce strongly in the presence of double-stranded nucleic acid and fluoresce weakly or not at all in the presence of single-stranded nucleic acid. A specific product will have a specific melting signature (i.e., a temperature transition range over which fluorescence decays as the duplex transitions from fully double stranded to fully single stranded), so nucleic melting can be used to confirm the identity of a nucleic acid amplification product. Sequence variants will typically melt at a different temperature and/or have a different melt curve shape than the wild-type sequence and, as such, Tm shifts and melt shape changes can be used to detect the presence of sequence variance in an amplified nucleic acid.

In this context, the term “detecting” may include the detection and determination of known and unknown nucleic acid sequence variances, including, but not limited to, SNPs, base deletions, base insertions, sequence duplications, rearrangements; inversions, base methylations, the number of short tandem repeats; and in the case of a diploid genome, whether the genome is a homozygote or a heterozygote of the sequence variance, as well as the cis/trans positional relationship of two or more sequence variances on a DNA strand (haplotyping). Such nucleic acid sequence variances may be associated with a number of genotypic and phenotypic differences in an organism. Single nucleotide polymorphisms (SNPs) are by far the most common genetic variations observed in man and other species. In these polymorphisms, only a single base varies between individuals. The alteration may cause an amino acid change in a protein, alter rates of transcription, affect mRNA spicing, or have no apparent effect on cellular processes. Sometimes when the change is silent (e.g., when the amino acid it codes for does not change), SNP genotyping may still be valuable if the alteration is linked to (associated with) a unique phenotype caused by another genetic alteration. In a specific example, nucleic acid sequence variances (e.g., SNPs) may be associated with an antimicrobial resistance (AMR) marker. Such nucleic acid sequence variances may be associated with detectable Tm shifts that can be used of sequence variants. “High resolution” melting and techniques for detection of mutations are described in greater detail in U.S. Pat. No. 9,657,347 (see, e.g., Example 19), U.S. Pat. No. 9,290,663, and U.S. Pat. Pub. No. 2018/0066137, the entireties of which were incorporated by reference elsewhere herein.

In one aspect of the present invention, a method is provided that requires only standard PCR reagents, primers, and the simple addition of a “saturating” double-stranded (ds) DNA binding dye prior to PCR. For purposes of this invention, a “saturating” dye is a dye that does not significantly inhibit PCR when present at concentrations that provide maximum fluorescence signal for an amount of dsDNA typically generated by PCR in the absence of dye, illustratively about 10 ng/μL. Although the dyes are identified by their compatibility with PCR at near saturating concentrations, it is understood that the dyes can be used at much lower concentrations. During or subsequent to amplification, the dyes may be used to identify the presence of known and unknown sequence variations by melting curve analysis in a similar fashion to when labeled primers are used. The identification of known and unknown sequence variations may be used for a variety of analyses, including mutation scanning and SNP genotyping. The term “scanning” refers to the process in which a nucleic acid fragment is compared to a reference nucleic acid fragment to detect the presence of any difference in sequence. A positive answer indicating the presence of a sequence difference may not necessarily reflect the exact nature of the sequence variance or its position on the nucleic acid fragment. The term “genotyping” includes the detection and determination of nucleic acid sequence variances.

Furthermore, while reference is made to PCR, other methods of amplification may be compatible with the dyes of this invention. Such suitable, procedures include strand displacement amplification (SDA); nucleic acid sequence-based amplification (NASBA); cascade rolling circle amplification (CRCA), Q beta replicase mediated amplification; isothermal and chimeric primer-initiated amplification of nucleic acids (ICAN); transcription-mediated amplification (TMA), and the like. Therefore, when the term PCR is used, it should be understood to include other, alternative amplification methods.

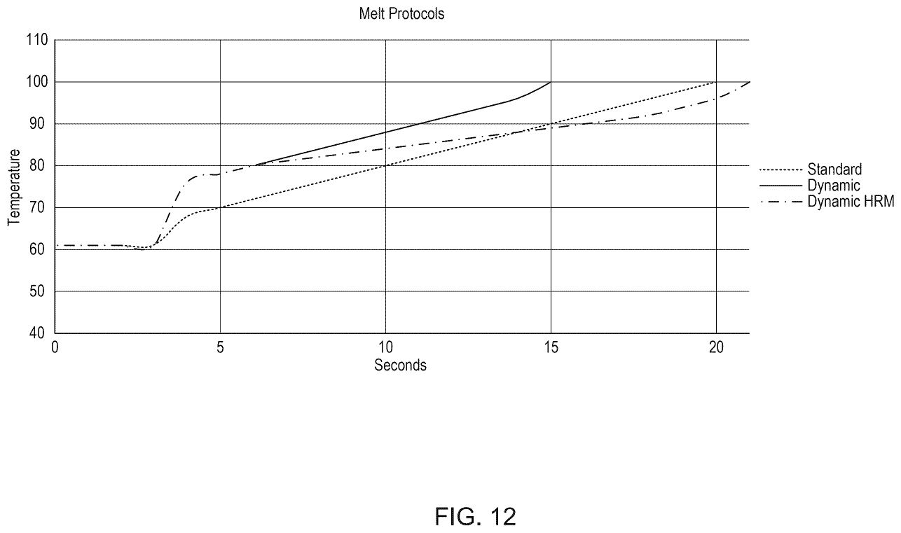

Referring now to FIG. 12, schematic melt procedures are shown that compare a standard melt protocol ( [symbol] ), a dynamic melt protocol ( [symbol] ) like the dynamic melt protocols described in detail elsewhere herein, and a dynamic “high resolution” protocol ( [symbol] ). For each procedure, there is a 3 s hold at 62° C. For the standard protocol, the temperature is then ramped to 68° C. at the max ramp rate of 12° C./sec. The melt is then executed at a standard (fixed) ramp rate of 2° C./sec up to 99° C. For the dynamic protocol, after the 3 sec hold, the temperature is ramped to 76° C. at a ramp rate of 12° C./sec. The ramp rate is then reduced to 2° C./sec to a temperature of 95 C°, then ramped again at 12° C./sec to 99° C. For the dynamic HRM protocol, after the 3S hold, the temperature is ramped to 76° C. at a ramp rate of 12° C./sec. The ramp rate is reduced to 1° C./sec from 76° C. to 92° C., then ramped to 12° C./sec to 99° C. In this illustrative example, the ramp rate for the melt portion is 1° C./sec. While this is illustrative, other “high resolution” melt ramp rates may be used such as, but not limited to, 0.01-2° C./sec, 0.1-1° C./sec, preferably no greater than 0.5° C./sec, more preferably no greater than 1° C./sec, and most preferably less than or equal to 2° C./sec. Choice of the ramp rate for detection of genetic variants will depend on factors such as the melt resolution needed and the time requirements of the assay. In general, the inventors in this case found a ramp rate of 0.5-1° C./sec to be sufficient in most cases.

In this illustrative example, the time for the standard melt protocol is 20 seconds, the dynamic protocol can be completed in 15 seconds, and the dynamic HRM protocol can be completed in 21 seconds. Implementation of the slower, “high resolution” ramp rate in the standard protocol would add ~30 seconds run time While implementation of the high resolution dynamic melt procedure does take about 6 seconds longer than the regular dynamic melt procedure in this example, the high resolution dynamic melt can be performed with only a 1 sec cost when compared to the standard protocol. While these are hypothetical procedures, this schematic illustrates the potential time savings of a dynamic high-resolution protocol versus a standard high-resolution protocol. As with the actual dynamic melt procedures described in detail elsewhere herein, actual protocols and actual time savings may depend on the melt characteristics of the amplicon being melted. Total run time cost for performing a high-resolution melting procedure will depend on the ramp rate and the number of individual melts performed.

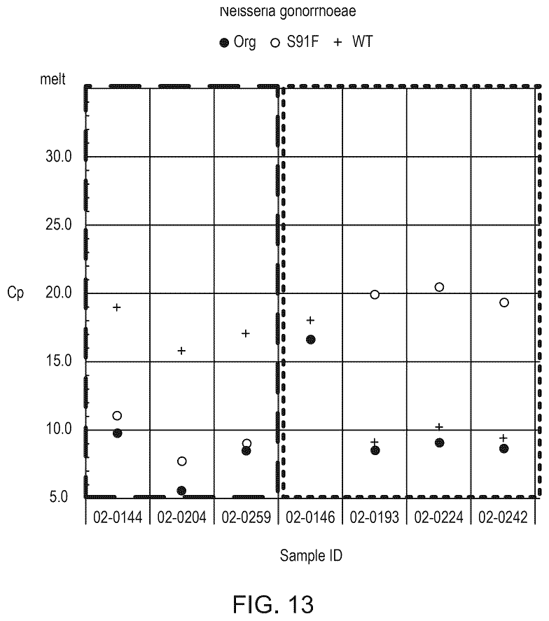

In some aspects of the present invention, high resolution melting procedures may be routinely used for the detection of certain nucleic acids. However, because high resolution melting procedures may be more time consuming, it may be desirable in other aspects of the present invention to use a first assay as a trigger to determine whether or not a melt should be performed in an assay for detecting a genetic variant. An illustrative example of such a triggering assay is illustrated in FIG. 13. FIG. 13 illustrates an N. gonorrhoeae organism assay (● Org) and a separate AMR assay targeted to a mutation prone region of the gyrA gene (∘ S91F and +WT). In the case of the left panel, the S91F assay (hollow dot) comes up closest to the organism (solid dot), whereas the WT assay is much later. This indicates that the isolate tested is resistant. In contrast, in the right panel, the assay closest to the organism is the WT assay, indicating that the sample contains wild-type N. gonorrhoeae. While FIG. 13 shows Cp differences between wild-type and mutant, such Cp differences are not a reliable way to identify wild-type and mutant isolates, in this case. In one aspect, the gyrA assay for identification of mutant or wild-type N. gonorrhoeae (i.e., antimicrobial resistant or antimicrobial sensitive N. gonorrhoeae) may only be performed if the Org assay is positive. This general approach may be applied to other assays for detection of genetic variances. This way, the extra time needed for detection of a genetic variance (e.g., an AMR assay) may only be needed if the organism is positive. In addition, because of potential cross-reactivity between different, related species in an organism family (e.g., Neisseria species), there may be instances where an assay for a genetic variance can give a false positive (the targeted region may be highly conserved among related organisms and, and such, the assay may not be selective). If the organism assay, which may be highly selective, is positive, then that may be used to trigger a melt for the genetic variance. Thus, the methods of the present invention described herein may include detecting presence of an organism via a first melt detection in first assay and, if the organism is detected as present in the first assay, performing a second melt detection in a second assay to detect presence of or absence of the genotype, sequence variant, or genetic mutation.

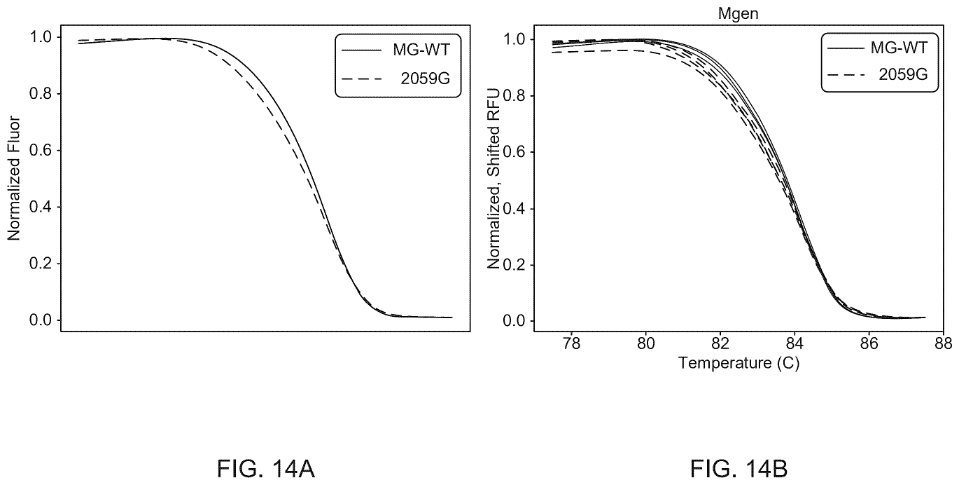

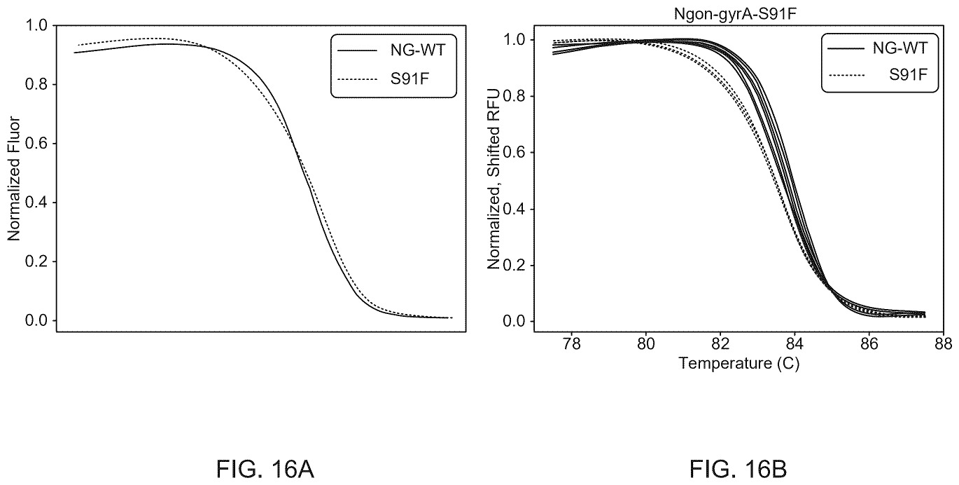

Referring now to FIGS. 14-16, illustrative examples are shown using a modified dynamic high-resolution melting procedure for detection of genetic variants in nucleic acids amplified in an experimental FilmArray test pouch. FIGS. 14-16 show melting curves for amplified wild-type and mutant nucleic acids from M. genitalium (23S) and N. gonorrhoeae (gyrA). The 23S mutations shown in FIG. 14 (A2059G) and FIG. 15 (A2058G) confer macrolide resistance on M. genitalium and the S91F mutation of gyrA shown in FIG. 16 confers ciprofloxacin resistance on N. gonorrhoeae. These and other antimicrobial resistance mutations are becoming more prevalent in many pathogenic bacteria due to the overuse of antibiotics. Accurate and routine identification of the presence of antimicrobial resistance markers is vital for antimicrobial stewardship and proper patient care. The methods described herein can be used for accurate and routine detection and identification of many types of genetic variances.

FIGS. 14A and 14B compare melt curves for wild-type ( [symbol] MG-WT) and A2059G ( [symbol] 2059G) mutant amplicons of the 23S ribosomal RNA gene of M. genitalium. FIG. 14A shows composite WT and mutant curves and FIG. 14B shows individual curves from individual amplification reactions and melts. In both cases, the melt curves for the WT and mutant are clearly distinguishable. The WT and mutant amplicons were melted with an initial ramp rate of 2° C./sec from 62° C. to 69° C., the ramp rate was reduced to 1° C./sec for the melt temperature range (referred to elsewhere herein as the ROI) from 69° C. to 94° C., then the ramp rate was increased to 2° C./sec from 94° C. to a final temperature of 99° C. These ramp rates are merely one example. For instance, persons of ordinary skill will understand that the initial and final ramp rates may be higher than 2° C./sec (e.g., 4-12° C./sec) and that other melt ramp rates may be used in the ROI such as, but not limited to, 0.01-2° C./sec, 0.1-1° C./sec, preferably no greater than 0.5° C./sec, more preferably no greater than 1° C./sec, and most preferably less than or equal to 2° C./sec. In general, slower ramp rates will increase the resolving power of the melts, at the expense of greater analysis time. Here, each of the samples were unambiguously genotyped with a melt ramp rate of 1° C./sec.

FIGS. 15A and 15B compare melt curves for wild-type ( [symbol] MG-WT) and A2058G ( [symbol] 2058G) mutant amplicons of the 23S ribosomal RNA gene of M. genitalium. FIGS. 15A and 15B are similar to FIGS. 14A and 14B. FIG. 15A shows composite WT and mutant curves and FIG. 15B shows individual curves from individual amplification reactions and melts. In both cases, the melt curves for the WT and mutant are clearly distinguishable. The melt parameters for FIGS. 15A and 15B are the same as those for FIGS. 14A and 14B. As with FIGS. 14A and 14B, persons of ordinary skill will understand that the ramping parameters may be changed to make the ramps faster or slower and/or to increase or decrease resolution of the melts. Here, each of the samples were unambiguously genotyped with a melt ramp rate of 1° C./sec.

FIGS. 16A and 16B compare melt curves for wild-type ( [symbol] NG-WT) and S91F ( [symbol] S91F) mutant amplicons of the gyrA gene of N. gonorrhoeae. FIG. 16A shows composite WT and mutant curves and FIG. 16B shows individual curves from individual amplification reactions and melts. In both cases, the melt curves for the WT and mutant are clearly distinguishable. The WT and mutant amplicons were melted with an initial ramp rate of 2° C./sec from 69° C. to 83.8° C., the ramp rate was reduced to 1° C./sec for the melt temperature range (referred to elsewhere herein as the ROI) from 83.8° C. to 91.8° C., then the ramp rate was increased to 2° C./sec from 91.8° C. to a final temperature of 99° C. As with FIGS. 14A-15B, persons of ordinary skill will understand that the ramping parameters may be changed to make the ramps faster or slower and/or to increase or decrease resolution of the melts. Here, each of the samples were unambiguously genotyped with a melt ramp rate of 1° C./sec.

The examples shown in FIGS. 14-16 illustrate situations where a dynamic melt protocol can be used to distinguish wild-type melts from mutant melts. The fact that this can be done in a FilmArray test pouch without significantly increasing assay time is a significant advance in the art. And while FIGS. 14-16 illustrate assays performed in a FilmArray test pouch, persons of ordinary skill will appreciate that these protocols can be adapted to essentially nucleic acid amplification and melting system. In addition, the examples shown in FIGS. 14-16 have relatively small temperature and shape differences in melting between wild-type and mutant, yet the differences can be clearly resolved using the dynamic melt procedure described herein. This suggests that the dynamic melt procedure described herein can routinely be used for detection of amplified nucleic acids containing mutations.

Although the invention has been described in detail with reference to preferred embodiments, variations and modifications exist within the scope and spirit of the invention as described and defined in the following claims.

What is claimed is:

1. A method for identifying which of a plurality of target nucleic acids is in a sample, comprising
providing the sample suspected of containing at least one of a plurality of target nucleic acids,
providing a plurality of sample wells, each sample well provided with a portion of the sample and primers for amplifying a target nucleic acid from a different one of the plurality of target nucleic acids, wherein each target nucleic acid has a melt temperature range that is characteristic for that target nucleic acid,
providing a fluorescent dye that produces a rising fluorescence signal in response to a rise in concentration of nucleic acids in the plurality of sample wells,
simultaneously subjecting the plurality of sample wells to amplification conditions for a selected number of cycles,
determining if one sample well exhibits positive nucleic acid amplification as evidenced by a rising fluorescence signal from the sample well during the amplification conditions, and
responsive to determining that the one sample well exhibits positive nucleic acid amplification, performing a melt detection configured to detect the target nucleic acid amplified in the one sample well, wherein the melt detection is defined by the melt temperature range characteristic for the target nucleic acid amplified in the one sample well.

2. The method of claim 1, wherein positive nucleic acid amplification is determined by the fluorescence signal in the sample well rising above a threshold, wherein the threshold is one of a crossing point (Cp), a Cp plus one or more additional cycles of amplification, Ct, relative florescence units rising above a threshold value, mathematical modeling, signal processing, and combinations thereof.

3. (canceled)

4. The method of claim 2, further comprising analyzing the fluorescent signal of the plurality of wells in real time to determine if amplification has occurred in a sample well and performing the melt detection if amplification is determined to have occurred in the well, wherein a temperature range for the melt detection is limited by a known melt temperature range for a nucleic acid of the target organism in the well.

5. The method of claim 1, further comprising not performing a melt detection if no sample well shows positive nucleic acid amplification.

6. The method of claim 5, wherein the plurality of sample wells includes one or more control wells and wherein a melt detection is not performed if only the one or more control wells show positive nucleic acid amplification.

7. The method of claim 1, wherein the melt detection comprises:
a first ramp rate during a first portion of the melt,
a second ramp rate during a second portion of the melt, and
a third ramp rate during a third portion of the melt,
the second ramp rate being slower than the first and third ramp rates and wherein the second portion of the melt is defined by the melt temperature range characteristic for the target nucleic acid amplified in the one sample well.

8. The method of claim 7, wherein the first ramp rate is greater than 4° C./sec, preferably in a range between 4° C./sec and 20° C./sec and more preferably in a range between 6° C./sec and 20° C./sec, the second ramp rate is less than 4° C./sec, preferably in a range between 0.01° C./sec and 4° C./sec, and the third ramp rate is greater than 4° C./sec, preferably in a range between 4° C./sec and 20° C./sec and more preferably in a range of 12° C./sec to 20° C./sec.

9. The method of claim 7, wherein the third ramp rate is faster than the first ramp rate.

10. The method of claim 8, wherein the second ramp rate is in a range of 0.01 to 4° C./sec, or, preferably, 0.01 to 2° C./sec.

11. (canceled)

12. The method of claim 10, wherein the second ramp rate is adapted to detect a nucleic acid melting signature indicative of a genotype, a sequence variant, a single nucleotide polymorphism (SNP), an antimicrobial resistance (AMR) marker, or a genetic mutation in a target nucleic acid that alters one or more of a nucleic acid melt temperature or a shape of a nucleic acid melting curve relative to the target nucleic acid without the genotype, sequence variant, or genetic mutation.

13-15. (canceled)

16. The method of claim 7, wherein a positive or negative call in a sample well determines whether a melt detection is performed and, if positive nucleic acid amplification is detected in the sample well, then the melt temperature range for the target amplicon expected for the sample well determines the temperature range of the second portion of the melt.

17. The method of claim 16, wherein the temperature range of the second portion of the melt is the melt temperature range for the target amplicon, the melt temperature range for the target amplicon+/−0.5° C. to 10° C., preferably the melt temperature range for the target amplicon+/−2° C. to 6° C.

18. The method of claim 1, wherein two or more sample wells show a fluorescent signal indicative of positive amplification.

19. (canceled)

20. (canceled)

21. The method of claim 20, wherein the melt detection comprises one of:
a first ramp rate during a first portion of the melt,
a second ramp rate during a second portion of the melt, and
a third ramp rate during a third portion of the melt,
the second ramp rate being slower than the first and third ramp rates and wherein the second portion of the melt is defined by the melt temperature ranges characteristic for the target nucleic acids amplified in the two or more sample wells, or
a first ramp rate during a first portion of the melt,
a second ramp rate during a second portion of the melt,
a third ramp rate during a third portion of the melt,
a fourth ramp rate during a fourth portion of the melt, and
a fifth ramp rate during a fifth portion of the melt,
the second and fourth ramp rates being slower than the first, third, and fifth ramp rates and wherein the second portion of the melt is defined by the melt temperature range characteristic for the target nucleic acid amplified in a first sample well of the two or more sample wells and the fourth portion of the melt is defined by the melt temperature range characteristic for the target nucleic acid amplified in a second sample well of the two or more sample wells.

22. The method of claim 21, wherein the second ramp rate is in a range of 0.05 to 4° C./sec, or the second and fourth ramp rates are each in a range of 0.05 to 4° C./sec, or wherein the second ramp rate or the second and fourth ramp rates are preferably no greater than 0.5° C./sec, more preferably no greater than 1° C./sec, and most preferably no greater than 2° C./sec.

23-27. (canceled)

28. A method for identifying which of a plurality of target nucleic acids is in a sample, comprising
providing the sample suspected of containing at least one target nucleic acid of the plurality of target nucleic acids,
providing a plurality of sample wells configured for amplification of the plurality of target nucleic acids, wherein each sample well of the plurality of sample wells comprises a pair of primers for amplification of one of the plurality of target nucleic acids, and wherein each target nucleic acid has a characteristic melt temperature range,
providing a fluorescent dye that produces a rising fluorescence signal in response to a rise in concentration of nucleic acid in the plurality of sample wells,
distributing the sample amongst the plurality of sample wells such that each sample well contains a portion of the sample,
simultaneously subjecting the plurality of sample wells to amplification conditions, wherein the amplification conditions include repeated thermal cycles each comprising a primer annealing step, a primer elongation portion, and a nucleic acid denaturation step,
acquiring during the thermal cycles the fluorescence signal in each of the plurality of sample wells,
determining that the amplitude of the fluorescence signal of one sample well configured for amplification of one target nucleic acid is greater than a threshold value, the threshold being greater or equal than a limit-of-detection value for concluding a rise in the concentration of nucleic acid in the sample well, and
responsive to determining that the amplitude of the fluorescence signal of the one sample well is greater than the threshold value, performing a melt detection configured to detect the target nucleic acid amplified in the one sample well, wherein the melt detection is defined by the melt temperature range characteristic for the target nucleic acid amplified in the one sample well for detection of the organism in the sample well,
wherein the threshold value is one of a crossing point (Cp), a Cp plus one or more additional cycles of amplification, Ct, relative florescence units rising above a threshold value, mathematical modeling, signal processing, and combinations thereof.

29. (canceled)

30. The method of claim 28, further comprising monitoring the fluorescent signal of the plurality of wells in real time to determine if amplification has occurred in at least the one well of the plurality of sample wells and performing the melt detection if amplification is determined to have occurred in at least the one well, wherein a temperature range for the melt detection is determined based on a known melt temperature range for a nucleic acid of the target organism in the at least the one well where amplification is determined to have occurred.

31. (canceled)

32. The method of claim 28, wherein the method does not include performing a set number of thermal cycles prior to performing a melt detection.

33. (canceled)

34. (canceled)

35. The method of claim 28, wherein the melt detection comprises:
a first ramp rate during a first temperature range of the melt,
a second ramp rate during a second temperature range of the melt, and
a third ramp rate during a third temperature range of the melt,
the second ramp rate being slower than the first and third ramp rates and wherein the second temperature range of the melt is defined by the melt temperature range of the target nucleic acid amplified in the one sample well having the fluorescence signal greater than the threshold value.

36-48. (canceled)

49. A method for determining presence of an organism in a sample, comprising
providing a sample suspected of containing at least one of a plurality of organisms,
providing a plurality of sample wells, each sample well provided with primers for amplifying a target nucleic acid from a different one of the plurality of organisms,
moving a portion of the sample into each of the plurality of sample wells,
simultaneously subjecting the plurality of sample wells to amplification conditions,
acquiring during the thermal cycles the fluorescence signal in each of the plurality of sample wells,
determining that the amplitude of the fluorescence signal of one sample well configured for amplification of one target nucleic acid is greater than a threshold value, the threshold being greater or equal than a limit-of-detection value for concluding a rise in the concentration of nucleic acid in the one sample well,
responsive to determining that the amplitude of the fluorescence signal of the one sample well is greater than the threshold value, performing a melt detection in a melt temperature window to detect the amplified target nucleic acid in the one sample well, wherein the melt temperature window is defined by the melt temperature range characteristic for the target nucleic acid amplified in the one sample well, and
responsive to performing the melt detection, determining the presence of the organism in the sample.

50. The method of claim 49, further comprising subjecting the plurality of sample wells to amplification conditions for a selected number of cycles, and if within selected number of amplification cycles a sample well shows positive nucleic acid amplification, then performing a melt detection, wherein the melt detection is defined by the melt temperature range characteristic for the target nucleic acid for detection of the organism in the sample well.

51. The method of claim 50, wherein the selected number of cycles of amplification is at least one but 15 or less, at least one but 20 or less, at least one but 25 or less, at least one but 30 or less, or at least one but 35 or less.

52. The method of claim 49, further comprising not performing a melt detection if no sample well shows positive nucleic acid amplification.

53-97. (canceled)

98. The method of claim 1, further comprising:
determining if at least a second sample well exhibits positive nucleic acid amplification as evidenced by a rising fluorescence signal from the sample well during the amplification conditions, and
responsive to determining that the at least second sample well exhibits positive nucleic acid amplification, performing a melt detection configured to detect the target nucleic acid amplified in the at least second sample well, wherein the melt detection is defined by the melt temperature range characteristic for the target nucleic acid(s) amplified in the at least second sample well.
